(19) United States
(12) Patent Application Publication
(10) Pub. No.: US 2017/0284922 A1
(43) Pub. Date: Oct. 5, 2017

(54) MICROFLUIDIC METHODS AND CARTRIDGES FOR CELL SEPARATION

(71) Applicant: SELEXIS S.A., Plan les Ouates (CH)

(72) Inventors: Nicolas Mermod, Plan-Les-Ouates (CH); Niamh Harraghy, Plan-Les-Ouates (CH); Xuan Droz, Plan-Les-Ouates (CH); Amar Rida, Lausanne (CH); Pierre-Alain Girod, Plan-Les-Ouates (CH); Alexandre Regamey, Plan-Les-Ouates (CH); Thierry Colombet, Lausanne (CH); Etienne Lancon, Plan-Les-Ouates (CH)

(21) Appl. No.: 15/509,203

(22) PCT Filed: Sep. 1, 2015
(86) PCT No.: PCT/EP2015/069907

§ 371 Date: Mar. 7, 2017

Related U.S. Application Data
(60) Provisional application No. 62/046,979, filed on Sep. 7, 2014.

(51) Int. Cl. G01N 15/10; G01N 33/543; B01L 3/00; C12N 5/071; G01N 33/569; G01N 33/574

(52) U.S. Cl. CPC G01N 15/1056; G01N 33/56966; G01N 33/57492; B01L 3/502715; B01L 3/502761; B01L 3/502723; C12N 5/0682; G01N 33/54333; G01N 33/54366; G01N 2015/1081; G01N 2015/1006; G01N 2015/0065

(57) ABSTRACT

The invention discloses a method for selecting cells depending on their level of displaying and preferably secreting a protein of interest from a population of heterogeneously expressing cells, comprising: (a) contacting said cells with magnetic beads coated with an affinity group to the said cells, (b) mixing the said magnetic beads with the cells to capture the cells displaying/secreting the protein of interest, (c) performing at least one washing step to remove the non-captured cells, and (d) recovering the cells to which that magnetic beads have bound.

### FIELD OF THE INVENTION

The invention relates to a method for selecting cells depending on their level of expression, preferably display, more preferably secretion, of a protein of interest from a population of heterogeneously expressing cells using magnetic beads. Further, the invention also relates to a microfluidic based, preferably disposable, sterile cartridge for cell selection based on their level of expression, preferably display, more preferably secretion, of a protein of interest and a method for handling magnetic beads within a microfluidic reaction chamber.

### BACKGROUND AND INTRODUCTION TO THE INVENTION

Constructing mammalian cell lines for the efficient production of therapeutic proteins has been greatly improved by the construction of more efficient DNA vectors and engineered cell lines (Girod et al., 2007, Galbete et al., 2009, Ley et al., 2013; LeFourn et al., 2014). Nevertheless, manual screening of cell lines, which is both time consuming and labor intensive, is still often performed to identify those with the best properties, for instance those that have the highest productivities. Thus, there is a need in the art to devise an automated procedure for the screening of top producer cell lines, ergo cell lines that produce the highest level of a transgene, from a large population of stably transfected cells. There is, in particular, a need to develop a method for the fast identification selection and/or sorting of CHO and other recombinant cells that express and preferably display, even more preferably secrete, high levels of, for example, therapeutic proteins.

There are some publications demonstrating the feasibility of using magnetic beads/particles to sort cells (e.g. with manually-operated tubes and magnets) in academic laboratories. Most of the described methods are slow and cumbersome, and have limited throughput and efficacy. Furthermore, manual procedures are difficult to adapt to GMP (good manufacturing practice) or GLP (good laboratory practice) facilities, and they are thus generally not used in biotech or pharmaceutical enterprises. Presented herein is the use of magnetic beads within a microfluidic setting to achieve preferably fully automated mammalian cell separation, based upon distinct expression levels of a given transgene expression product.

While the MACS device sold by Miltenyi Biotec® allows the removal of dead cells from cultures of mammalian cell lines using magnetic beads in combination with a magnetic material column operated under a strong permanent magnet, MACS does not allow for selective sorting of magnetic beads, and it does not allow for a sorting of high and low producer cells to preferably identify and select high producer cells. Alternative methods and apparatuses that rely on the labeling of high-producer cells with antibodies have been disclosed. The fast isolation of high producer cells may involve the use of fluorescence cameras that image cell colonies growing in soft agar and are combined with the robotic picking of highly fluorescent colonies. Examples are TAP's CellCelector™ for stem cell picking (Caron et al., 2009). Alternatively, Genetix's ClonePix™ relies on the formation of immuno-precipitates from the secreted proteins in semi-solid culture media, similarly coupled to cameras and a cell-picking arm. In these approaches the cells are not grown as free suspension but as clumps and are picked early during cloning, in particular, before stable expression may have established. The equipment involved has a relatively low throughput in that it is unable to analyze 100,000 transfected cells and more, which, however, is generally needed to find the most productive clones. In addition, the approaches are relatively slow, requiring days to be performed. The microfluidic-based approach, of the present invention, is designed to mitigate and/or address drawbacks of the prior art.

### SUMMARY OF THE INVENTION

In one embodiment, the present invention is directed at a sorting method for cells that display a protein of interest and, in certain embodiments, produce a transgene of interest, such as a therapeutic protein, preferably at a high level and optionally from a complex polyclonal population.

In certain embodiments, the present invention can identify high-producer cell lines using magnetic beads in an easy-to-use microfluidic system in a relatively short amount of time (e.g., less than 36 or 24 hours). In other embodiments, viable cells (e.g., high-producer cells) are sorted using a single use (disposable) cartridges in a consistently sterile environment, as required to achieve GMP compatible cell sorting.

The invention also concerns method for selecting cells depending on their level of expression of a protein of interest from a population of heterogeneously expressing cells using magnetic beads.

The invention is also directed at a method for identifying and, preferably selecting, cells displaying a protein of interest on their surface comprising:
(a) providing a sample comprising said cells;
(b) providing functionalized magnetic beads comprising one or more affinity groups, and optionally carrier beads, wherein said affinity group(s) is adapted to bind cells displaying the protein on their surface;
(c) mixing the cells with said functionalized magnetic beads and optionally with said carrier beads,

wherein said affinity group of the beads binds cells displaying said protein on their surface to produce magnetically-labeled cells (MLCs) having a magnetic label,
(d) separating, e.g. in at least one washing step, non-magnetically labeled cells from said MLCs, and
(e) identifying and, preferably selecting cells displaying the protein on their surface.

The protein of interest may be a marker protein or a transgene expression product (TEP).

The cells may be recombinant cells and the sample may comprise the recombinant cells that were transfected with a transgene, wherein the protein of interest may be a transgene expression product (TEP); and wherein the MLCs may lose their magnetic label over a time interval after binding to the affinity group and wherein the MLCs may be identified, and preferably selected, based on the time interval.

The recombinant cells secreting the TEP may be separated from recombinant cells displaying, but not secreting, the TEP based on said time interval.

The MLCs that lose the magnetic label in less than 1, 2, 3, 4, 5, 6, 7, 8, 9, 10, 11, 12, 13, 14, 15, 16, 17, 18, 19, 20, 21, 22, 23 hour(s) after binding, in less than 24, in less than 36, in less than 48, in less than 36, in less than 60, in less than 72, in less than 84 or in less than 96 hours after binding may be selected.

The protein of interest may be a marker protein identifying a stem cell, in particular a cancer stem cell (CSC) or a circulating tumor cell.

The affinity group of the magnetic beads may bind the protein directly.

At least one linking molecule may bind the affinity group and the protein, linking the magnetic beads to the protein. The linking molecule may be an antibody or fragment thereof, which may be biotinylated.

The cells may be mixed at a temperature above 20, 24, 26, 28, 30, 32, 34 or 36 degrees.

The mixture may be a mixture of functionalized beads (capture beads) and carrier beads and the mixture may be in a reaction chamber.

The method may further comprise applying an external magnetic field having an amplitude and a polarity to said reaction chamber, wherein, in said external magnetic field, mixing of the capture beads and the cells displaying the protein may be promoted by said carrier beads.

The magnetic beads may be manipulated using a magnetic field having a polarity and amplitude that varies in time. The variation of the said magnetic field may involve a variation of frequency ranging between 0.1 to 1000 cycles per second. Cells selection may be achieved by controlling the frequency and the amplitude of the applied magnetic field. Cell selection may also be achieved by controlling the magnetic beads and cell mixing time. Cell selection may be further controlled by one of more parameters that include the number of washing steps, the nature of the magnetic beads, and the cell mixing time during the washing steps.

The selected cells may have a level of protein expression, display or secretion that is at least 10% higher than the cells present in the original population. selected cells have a level of protein expression, display or secretion may preferably be 20%, 40%, 60%, 80%, or more preferably over 90% higher than the cells present in the original population. Cell may also be selected on the basis of their lower protein expression and the selected cells may have a level of protein expression is at least 10% lower than the cells present in the original population. The selected cells may have a level of protein expression that is preferably 20%, 40%, 60%, 80%, or more preferably over 90% lower than the cells present in the original population.

The capture beads may be superparamagnetic beads and the carrier beads may be ferromagnetic beads.

The ratio of capture beads to said carrier beads may be between 2:1 and 50:1, 5:1 and 25:1, preferably between 8:1 and 12:1 or around 10:1.

The amplitude and/or the polarity may be changed to define successive operation modes, wherein said mixing in (c) may be performed in a mixing mode and said separating in (d) may be performed in a bead separation mode.

The cells may be recombinant cells and the protein expressed on the surface may be a TEP and the identifying in (e) is performed by eluting the cells from the reaction chamber that lose their magnetic label (ergo separate from the magnetic bead) in less than 48 hrs, preferably less than 36 or 24 hrs after binding.

In the mixing and bead separation mode, the magnetic device(s) may operate in a circular or alternating mode at 1 Hz-1000 Hz and 0.1 to 10000 mA, preferably 40 to 500 Hz and at 200-500 mA.

The mixing mode and/or bead separation mode may each last less than 60 seconds.

The invention is also directed at a cartridge for selecting cells based on their level of display, and preferably secretion (release from a surface of a cell; shedding), of a protein, such as a TEP, from a population of cells comprising the cells displaying, preferably secreting said protein, comprising:
a. microfluidic channels,
b. a reaction chamber for mixing magnetic beads in suspension, wherein the reaction chamber has at least one inlet and at least one outlet channel for introducing and removing a fluid into and from, respectively, said reaction chamber,
c. a cell sample container in fluid communication with the reaction chamber through the inlet channel,
d. at least one washing reagent container in fluid communication with the reaction chamber through the inlet channel,
e. a waste container in fluid communication with the reaction chamber through the outlet channel,

wherein, each container of c. to d. is further in communication through one of the microfluidic channels to a venting pore comprising an air filtering element.

The invention is also directed to an integrated system for selecting cells, e.g. recombinant cells, based on their level of display, and preferably secretion (release from a surface of a cell; shedding), of a protein, e.g., a TEP, expressed on the surface of the cells, from a population of cells comprising cells displaying, preferably secreting, said protein, wherein the system comprises a cartridge comprising:
a. microfluidic channels,
b. a reaction chamber for mixing magnetic beads in suspension; wherein the reaction chamber has at least a first inlet and at least a second outlet channel for introducing and removing a fluid into and from said reaction chamber,
c. a cell sample container in fluid communication with the reaction chamber through the inlet channel,
d. at least one washing reagent container in fluid communication with the reaction chamber through the inlet channel,
e. a waste container in fluid communication with the reaction chamber through the outlet channel, wherein each container of c. to d. is further in communication through one of the microfluidic channels to an venting pore comprising an air filtering element;
f. one or more devices that create a controllable magnetic field (magnetic field devices=MFDs), in particular one or more electromagnets, arranged around or at the reaction chamber;
g. data processing equipment (e.g. a computer) configured to adjust a magnetic field created by the MTD(s) within the reaction chamber via frequency and/or amplitude adjustments, wherein each frequency and/or amplitude adjustment defines an operation mode within the reaction chamber.

The data processing equipment may be configured to set a succession of said operation modes comprising a mixing mode, a capture mode, an immobilization mode, a bead separation mode and/or a recovery mode.

The data processing equipment may be adapted to set the MFDs to operate:
in a circular or alternating mode at 1-1000 Hz, preferably 40 Hz-500 Hz and at 0.1 to 10,000 mA, preferably 200-500 mA during the mixing and bead separation mode, wherein, e.g., the circular mode may switch between clockwise and counterclockwise;
in circular or alternating mode at a frequency and amplitude lower than in the mixing mode, such as at 0.5 to 40 Hz and at 300 to 600 mA, during the capture mode;
at 0 Hz and at an amplitude, such as at 300 to 600 mA, during the immobilization mode; and
at an, relative to the immobilization mode, increased frequency, such as between 40 Hz-500 Hz and at a lowered amplitude, such as at 30-300 mA during the recovery mode.

The reaction chamber of the system or cartridge may comprise a mixture of carrier and capture beads.

The cartridge may further include a recovery container for receiving magnetically labeled cells, preferably magnetically labeled recombinant cells from the reaction chamber.

The cartridge or system may further comprise at least one second inlet and at least one second outlet channel in fluid communication with said reaction chamber, wherein the second inlet channel diverges off the at least one first outlet channel and the second outlet channel diverges off the at least one first inlet channel, wherein the recovery container is in fluid communication with the reaction chamber through the second inlet channel and the second outlet channel is connected to a further venting pore comprising an air filtering element.

The air venting pore of the recovery container may be connected to a pump for recovering the magnetically labeled cells within the reaction chamber by pumping air through the venting pore of the recovery container so that the reaction chamber content is flushed into the recovery container through an inlet channel.

The reaction chamber volume may be between 10 μl and 500 μl.

The cartridge may be self-contained and/or disposable.

The invention is also directed at a kit comprising, in one container, a cartridge as described herein, wherein the reaction chamber may comprise capture beads and carrier beads (which may alternatively be contained in a further container), and, in a separate container, instructions of how to use the capture beads and carrier beads in the cartridge.

The capture beads may be superparamagnetic beads and the carrier beads may be ferromagnetic beads, wherein the ratio of superparamagnetic beads to ferromagnetic beads is between 2:1 and 50:1.

The invention is also directed at cells identified and preferably selected via the methods, systems and/or cartridges described herein.

The invention is also directed at an isolated population of cells comprising, preferably recombinant cells secreting a transgene expression product, at a level of more than 20, 40, 60, 80 pcd, wherein the isolated population of claims does not contain more 40% of a original cell population from which the isolated cell population was isolated.

The invention also includes the use of mammalian cells disclosed herein as therapeutic cells, including, but not limited to gene therapy or regenerative medicine use.

The transgene secreted may be a therapeutic protein.

The time interval between the mixing the cells with said functionalized magnetic beads and optionally with said carrier beads, and the identifying and, preferably selecting cells displaying the protein on their surface may be less than 1 hour, less than 30 minutes, less than 20 minutes, less than 15 minutes or less than 10 minutes.

The subject matter of the claims and all claimed combinations is incorporated by reference in this description and remains part of the disclosure event if claims are abandoned.

### BRIEF DESCRIPTION OF THE DRAWINGS

The objects and features of the present invention are set forth with particularity in the appended claims. The present invention, both as to its organization and manner of operation, together with further objects and advantages, may best be understood by reference to the following description, taken in connection with the accompanying drawings, wherein

FIG. 1 is a schematic presentation showing the generation of cells with various immunoglobulin production levels. CHO-M cells were co-transfected with expression vectors for immunoglobulin gamma (IgG) and an antibiotic selection marker, as well as a plasmid encoding eBFP2. Polyclonal populations stably expressing various levels of IgG were sorted by FACS on the basis of BFP and surface IgG display. IgG secretion of selected cell clones were validated by ELISA.

FIG. 2 shows diagrams of GFP- or BFP-labeled reference cells mediating various

IgG display and secretion levels: CHO-M-derived cell clones displaying various levels of cell surface IgG, but with variables levels of IgG secretion were selected by FACS as reference cell populations. BFP-labeled median displayer BS2 cells, high displayer BLC cells and very high displayer BHB cells are compared to the GFP-labeled F206 very high producer cell clone. The IgG displayed at the cell surface was labeled with APC-conjugated anti-IgG antibodies, prior to flow cytometry analysis (A). The IgG titter produced by parallel cultures of the indicated cell clones (B), or their specific productivity in pictogram per cell and per day (C), were determined by ELISA assays of the IgG secreted into the cell culture medium.

FIG. 3 is a schematic presentation showing the principles of the manual capture of mixed cell populations. A mix of IgG-expressing and non-expressing cell populations at 1×10⁷ cells/mL was incubated with KPL biotin-conjugated anti-human IgG antibody to a final concentration of 5 μg/mL for 20 min. After a 5 min wash with 1× PBS followed by centrifugation of the cells at 1000 rpm, the pre-labeled cells were subsequently incubated with streptavidin-coated superparamagnetic beads for 30 min. A hand-held magnet allowed the separation of beads—captured IgG—displaying cells from non-expressing cells. The whole process was performed at room temperature.

FIG. 4 is a schematic presentation showing a demonstration of the manual enrichment of expressing cells from a mixed population of non-expressing cells. Manual capture recovered cells after each wash was put in cell culture, and grown without selection for 10 days, prior to IgG display assessment. 3 washes were efficient to remove most of the non-expressing cells, therefore only IgG positive cells were retained.

FIG. 5 is a schematic presentation of the cartridge design for automated enrichment of highly expressing cells from mixed cell populations using the MagPhase™ equipment. A schematic drawing (A), as well as an actual photograph (B), of the cartridge are shown to illustrate the arrangement of some of its elements.

FIG. 7 is a schematic presentation showing the type of magnetic microparticles used for automated enrichment of expressing cells from mixed cell populations.

FIG. 6 is a schematic presentation of the choice of magnetic microparticles for automated enrichment of expressing cells from mixed cell populations.

FIG. 7 is a presentation of the Manual cell capture with 2.8 μm superparamagnetic beads. A suspension of IgG-expression F206 cells (1×10⁷ cells/mL) was incubated with KPL biotinylated anti-human IgG antibodies for 20 min, before being subjected to a 30 min incubation with 30 μL of superparamagnetic beads. CHO cells bound to superparamagnetic beads are as indicated.

FIG. 8 is a presentation of the Manual cell capture with 2.0 μm ferromagnetic beads. A suspension of IgG-expression F206 cells (1×10⁷ cells/mL) was incubated with KPL biotinylated anti-human IgG antibodies for 20 min, before being subjected to a 30 min incubation with 30 μL of superparamagnetic beads. CHO cells bound to superparamagnetic beads are as indicated. CHO cells bound to ferromagnetic beads cannot be released into cell culture, as they formed aggregates.

FIG. 9 is a schematic presentation of the MagPhase™ automated cell capture with combination of superparamagnetic and ferromagnetic beads: mixing mode. The high frequency mixing mode is used for cell capture or washing steps. The two types of beads are dissociated and mix separately at the following conditions: 100-150 Hz and 200-300 mA, depending on the type of microbeads used, for 10 s. The electromagnets are activated consecutively in an circular fashion, with 1 second of clockwise rotation (1-2-3-4), 1 s of anticlockwise rotation (4-3-2-1), followed by 10 s of clockwise rotation, to achieve optimal mixing. The ferromagnetic beads (Black) circulate around the chamber near the walls, while superparamagnetic beads (grey) are dispersed all over the chamber to be incubated the cells for binding, or to mix in the washing buffer.

FIG. 10 is a schematic presentation of the MagPhase™ automated cell immobilization with combination of superparamagnetic and ferromagnetic beads: capture mode. Very high magnetic force (400 mA) and low frequency (1 Hz) are used in an anticlockwise rotation mode for 10 s, to allow ferromagnetic beads to circulate slowly all around the chamber, catching superparamagnetic beads and possibly associated cells.

FIG. 11 is a schematic presentation of the MagPhase™ automated cell immobilization with combination of superparamagnetic and ferromagnetic beads: immobilization mode. Associated superparamagnetic and ferromagnetic microbeads are immobilized on chamber walls at very high magnetic force (400 mA) and null frequency (0 Hz) during 10 s, allowing to pump in cells in suspension or various wash buffers. In this operation, the electromagnet are operated in a fixed mode (for instance 1 and 4 as negative poles, 2 and 3 as positive poles).

FIG. 12 is a schematic presentation of the MagPhase™ automated cell elution and recovery with combination of superparamagnetic and ferromagnetic beads: bead separation mode. Superparamagnetic and ferromagnetic beads are first separated by tumbling (100-150 Hz and 200-300 mA), and electromagnets are then operated as in the mixing mode (see caption for FIG. 9).

FIG. 13 is a schematic presentation of the MagPhase™ automated cell elution with combination of superparamagnetic and ferromagnetic beads: recovery mode. After beads separation during the previous step (FIG. 12), a medium frequency and magnetic force step (100 Hz and 100 mA) is applied for 3 s, where electromagnet are operated in the ‘beads immobilization’ mode (see FIG. 11), except that the positive and negative magnetic poles are switched with a 100 Hz frequency (to be confirmed). This magnetic force quickly corners the ferromagnetic but not the superparamegnatic beads on the chamber walls. The mid-range frequency keeps the superparamagnetic beads in the middle of the chamber, allowing to pump them out for collecting bound cells. The superparamagnetic beads are eluted by pumping air in the chamber during 4.5 s at a rate of 30 μl/s.

FIG. 14 is a presentation of the identification of MagPhase™ optimal magnetic field strength and field oscillation frequency to separate high (F206) and medium (BS2, BLC) producer cells with superparamagnetic and ferromagnetic beads. Microbeads and MagPhase™ operation conditions were as described in FIG. 9-13, except that 3 washing steps were performed at various frequencies and magnetic field intensities before the recovery mode, with the indicated conditions. This allowed the identification of the optimal conditions, whereas increased frequencies and/or magnetic fields (indicated by ‘Fast’ and ‘Strong’, respectively) yielded lower enrichment of the highly expressing F206 cells. The ratio of high vs. medium expressor cells in the input population was set to approximately 50:50 of F206:BS2 cells (A) or 30:70 of F206:BLC cells (B). Recovered cells were quantitated by fluorescence microscopy.

FIG. 15 is a presentation of the identification of the MagPhase™ optimal settings for cell incubation time. Mixing beads with cells for cell capture at 120 Hz, 300 mA for different times (2 s to 5 min) and 3 washing steps were performed at 120 Hz, 300 mA for 10 s. 1 μL of Chemicell SiMAG 1.0 μm beads and 20 μL Dynabeads MyOne T1 beads were preloaded in the mixing chamber. F206 and CHO-M cells were mixed at 10:90 ratio, and the cell mix was labeled with the biotinylated anti-IgG KPL antibody prior to MagPhase™ operations. Recovered cells were analyzed under fluorescence microscope.

FIG. 16 is a presentation of identification of the MagPhase™ optimal settings for ferromagnetic and superparamagnetic beads ratio. 1 or 2 μL of Chemicell SiMAG 1.0 as well as 5 μL, 10 μL, 20 μL or 30 μL of MyOne T1 Dynabeads were pre-loaded in the mixing chamber. F206 and CHO-M cells were mixed at 10:90 ratio and labeled with the biotinylated antibody. Recovered cells were analyzed under fluorescence microscope.

FIG. 17 is a presentation of IgG-expressing cell enrichment with a combination of superparamagnetic and ferromagnetic beads using MagPhase™ optimal automated settings. Indicated cell population mix was pre-incubated with KPL biotinylated anti-human IgG antibodies to a final concentration of 5 μg/mL. MagPhase-based cell separation was performed using 20 μL of superparamagnetic beads (MyOne T1 Dynabeads, Streptavidin coated, 1.0 μm) and 2 μL of ferromagnetic beads (Chemicell FluidMAG/MP-D, 5.0 μm, starch coated) preloaded into the mixing chamber. The optimized MagPhase™ steps and parameters were: 1. Mixing for cell capture at 120 Hz, 300 mA for 10 s, 2. Beads capture at 1 Hz, 400 mA for 10 s, 3. Beads immobilization at 0 Hz, 400 mA for 10 s, 4. 3 wash cycles were performed, and 5. Recovery at 100 Hz, 100 mA. The washing cycles consisted of the input of 100 μl of PBS buffer followed by mixing mode, beads capture and beads immobilization steps as above. Recovered cells were analyzed under fluorescence microscope.

FIG. 18 is a presentation of the MagPhase™ automated separation of high (F206) from medium (BS2), high (BLC) and very high (BHB) IgG displayer cells with superparamagnetic and ferromagnetic beads. Microbeads, cells preparation and MagPhase™ operation conditions were the same as described in FIG. 17. Recovered cells were analyzed under fluorescence microscope.

FIG. 19 is a presentation of the comparison of MagPhase™ automated capture and manual capture. Indicated cell population (F206 and CHO-M cells mixed to 10/90 ratio (A); F206 and BS2 cells mixed to 40/60 ratio (B)) were pre-incubated with KPL biotinylated anti-human IgG antibodies to a final concentration of 5 μg/mL. MagPhase-based cell separation was performed using 20 μL of superparamagnetic beads (MyOne T1 Dynabeads, Streptavidin coated, 1.0 μm) and 2 μL of ferromagnetic beads (Chemicell FluidMAG/MP-D, 5.0 μm, starch coated) preloaded into the mixing chamber. The MagPhase™ procedure and manual capture procedure were carried out as described in FIG. 17 and FIG. 3, respectively. Recovered cells were analyzed under fluorescence microscope.

FIG. 20 depicts the sterile capture and enrichment of IgG-expressing cells using first-generation MagPhase™. F206 and CHO-M input cells were mixed to a ratio of 10:90 to 20:80, and a MagPhase™ capture process was performed as described in FIG. 17, using sterilized MagPhase™ cartridges. (A) MagPhase™ captured cells were separated from eluted beads on Day 1 after capture and they were put in culture without antibiotic selection for 16 days prior to IgG display analyses, in parallel to an aliquot of input cells cultivated as a control. (B) Cells were treated as for panel A, except that the cells were cultivated in presence of the CB5 feed prior to sorting with MagPhase™ and cells not eluted from the beads at Day 1 were recovered at day 3 post-sorting. Captured cells and the control cells were labeled with an APC-conjugated anti-IgG antibody, to stain the F206 cells that express and display the IgG, and subsequently analyzed by flow cytometry. (C) Manual and MagPhase™-mediated sorting were performed in parallel with cells cultured or not in presence of CB5 prior to performing the sorting. Recovered cells were analyzed by fluorescence microscopy. These results represent the average of the fold-enrichment of F206 cells obtained from 3 independent experiments.

FIG. 21 depicts the sterile MagPhase™ capture and enrichment of cells that both express and secrete high levels of IgG, as eluted from the magnetic beads one Day 1 following MagPhase™ separation. The MagPhase™-captured cells of FIG. 20B, separated at Day 1 or Day 3 following capture, as well as an aliquot of input cells as control, were put in culture without antibiotic selection for 10 days prior to IgG secretion analysis. The specific productivity was expressed in pg of IgG secreted per cell and per day (pg/cell/day).

FIG. 22 is a presentation of the sterile MagPhase™ capture and enrichment of cells that both express and secrete high levels of IgG from a polyclonal population. The polyclonal cell population cultured in the absence of the CB5 feed was sorted using MagPhase™ as described in FIG. 21. Captured cells eluted from the magnetic beads at Day 1 and Day 4 post-sorting, as well as an aliquot of input cells as control, were placed in culture with CB5 but without antibiotic selection for 14 days, prior to assessing IgG display at the cell surface and IgG secretion in the cell supernatant by ELISA assays. (A) Percentage of the IgG positive cells, distinguishing low, medium and high displayer cells. (B) Specific productivity of IgG secretion in the supernatant of the cells eluted at Day 1 or Day 4 post sorting (pg/cell/day).

FIG. 23 is a presentation of the sterile MagPhase™ sorting to enrich cells highly expressing and secreting a therapeutic IgG from a polyclonal population, using different monoclonal antibodies (mAbs). MagPhase™ capture was performed as described in FIG. 17, using Mabtech or Acris mAbs labeled C_MF polyclonal cells as input. MagPhase™ captured cells separated on Day 1 of capture as well as an aliquot of input cells as control cells were split into 2 halves, respectively. Each half of cells was put in culture with or without CB5 and without antibiotic selection for 14 days prior to IgG display analyses. Cell culture supernatant was sampled on the same day of IgG display analyses. IgG titer in the supernatant samples were analyzed by ELISA for further calculation of specific productivity. (A) Percentage of the IgG positive cells, when cultured without CB5. (B) Percentage of the IgG positive cells, when cultured with CB5. (C) Specific productivity of IgG (pg/cell/day).

FIG. 24 is a presentation of the enrichment of IgG-expressing F206 cells from non-expressing cells using second generation and optimized MagPhase™ equipment and single use sterile cartridges. F206 and CHO-M cells were mixed to a ratio of 20:80 as input. Old MagPhase™ capture was performed as described in FIG. 17. 160 μL of biotinylated antibody labeled cells and 1360 μL of 1× PBS solution were loaded in the sample tube and wash solution tube of new MagPhase™ cartridge, respectively. The script run on new MagPhase™ had the same steps as the old MagPhase™ script, except pumped liquid volumes were adapted for the new MagPhase™, and amperage is half of that in the script of old MagPhase™. Both MagPhase™ captured cells were separated at Day 1 and Day 6 of capture as well as an aliquot of input cells as control cells were put in culture without antibiotic selection for 6 days. Recovered cells and control cells were analyzed by fluorescence microscopy. These results are the mean values obtained for 3 independent experiments. (A) Percentage of the IgG positive cells in captures using the KPL antiserum. (B) Percentage of the IgG positive cells in captures using Mabtech mAbs.

FIG. 25 is a schematic representation of the fluidic cartridge according to a preferred embodiment of the invention as used in FIG. 24. (Cartridge (1), reaction chamber (2), reaction chamber has inlet (3in) and outlet (3out) channels (for introducing and removing the liquid medium into and from said reaction chamber), cell sample container (4), washing reagent container (5), air venting pore (7), air filtering element, recovery container (9) (for receiving the selected cells from the reaction chamber (2)), second inlet and outlet channels (10in, 10out) (which are diverging branches of the first outlet and inlet channels (3out, 3in) respectively); the recovery container (9) is in fluid communication with the reaction chamber through the second inlet channel (10in) and the second outlet channel (10out) which is connected to an venting pore (7recovery) comprising an air filtering element (8).

FIG. 26 shows an analysis of cell populations sorted with a MagPhase™ device. Analysis was performed using a ClonePix™ imaging equipment that indicates the amount of released Trastuzumab antibody by each analyzed CHO cell colony (=clone). The identification of clones with extremely high productivities was possible.

FIG. 27 is a flow diagram showing the successive operation modes of a microfluidic device, here a MagPhase™ device with cartridge, as executed by the data processing equipment of the present invention.

### DETAILED DESCRIPTION OF VARIOUS AND PREFERRED EMBODIMENTS OF THE INVENTION

In various embodiments of the invention, magnetically susceptible beads (also referred to herein as “magnetic bead”, “magnetic particles, “magnetic microbeads” or just “microbeads”) are used. The magnetic beads may be made of any material known in the art that is susceptive to movement by a magnet (e.g., permanent magnet, but preferably an electromagnet). They are capable of producing high magnetic field gradients when magnetized by an external magnetic field.

In some embodiments of the invention, the beads are completely or partially coated, ergo functionalized, with an affinity group. Such an affinity group might be a ligand that directly attaches to a protein (receptor/marker protein, e.g. for stem cells) on the surface of a cell or to another surface expressed moiety, such as a transgene product, e.g., a therapeutic protein. The affinity group might also be a polymer material, an inorganic material or a protein such as streptavidin, which has high affinity to other molecules such as the vitamin biotin which is often used as a label for antibodies. The beads may comprise a ferromagnetic, paramagnetic or a superparamagnetic material or a combination of these materials. The magnetic beads may comprise a ferrite core and a coating. However, the magnetic beads may also comprise one or more of Fe, Co, Mn, Ni, metals comprising one or more of these elements, ordered alloys of these elements, crystals made these elements, magnetic oxide structures, such as ferrites, and combinations thereof. In other embodiments, the beads may be made of magnetite (Fe₃O₄), maghemite (γ-Fe₂O₃), or divalent metal-ferrites.

In certain embodiments of the invention, the magnetic beads comprise a non-magnetic core, for example, of a material selected from the group consisting of polystyrene, polyacrylic acid and dextran, upon which a magnetic coating is placed. There are different types of beads, wherein the “types” of beads are distinguished based on their magnetic behavior:

A “paramagnetic” bead is characterized by low magnetic susceptibility with rapid loss of magnetization once no longer in a magnetic field.

“Ferromagnetic” beads have high magnetic susceptibility and are capable of conserving magnetic properties in the absence of a magnetic field (permanent magnetism). Ferromagnetism occurs, e.g., when unpaired electrons in a material are contained in a crystalline lattice thus permitting coupling of the unpaired electrons. Preferred ferromagnetic materials include, but are not limited to, iron, cobalt, nickel, alloys thereof, and combinations thereof.

So-called “superparamagnetic” beads are characterized by high magnetic susceptibility (i.e. they become strongly magnetic when they are placed in a magnetic field), but like paramagnetic materials, they lose their magnetization quickly in the absence of the magnetic field. Superparamagnetism can be obtained in ferromagnetic materials when the size of the crystal is smaller than a critical value. Superparamagnetic beads present the dual advantages of being capable of being subjected to strong attraction by a magnet, and of not clumping together in the absence of a magnetic field. In particular, the property of not clumping together will preferably allow cells attached to the beads to remain viable.

Beads behaving as different types (e.g. ferromagnetic and superparamagnetic) depending on the surrounding condition have been disclosed elsewhere, e.g., in U.S. Pat. No. 8,142,892 which is incorporated herein by reference in their entirety and can be used as a “type” of magnetic beads in the context of the present invention. Other types of beads are disclosed, e.g., in US Patent Application 2004/0018611, which is incorporated herein by reference in its entirety.

In a preferred embodiment, the magnetic beads are very small, typically about 0.1 to 500 μm, preferably between 0.1 and 100 μm, more preferably between 0.2 and 50 μm, between 0.2 and 20 μm, between 0.2 and 10 μm and 0.2 and 5 μm. The relationship between the particle size and the magnetic force density produced by the particles in response to an external magnetic field is given by the equation:

fₘ=B₀I grad H I=B₀ M/a

where fₘ is magnetic force density, B₀ is the external magnetic field, I grad H I is the expression for the local gradient at the surface of a magnetic bead, M is the magnetization of the matrix element, and a is the diameter of the bead. Accordingly, the smaller the magnetic beads, the higher the magnetic gradient. Smaller beads will produce stronger gradients, but their effects will be more local.

In one embodiment, the magnetic beads are of non-uniform size, in others they are of uniform size. Generally, any shape of beads may be used, that is, any shape having an angle or curvature will form gradients. While smaller magnetic beads produce higher magnetic force density, larger beads produce a magnetic field gradient that reaches further from their surface. Generally, this is attributable to the higher radius of curvature of the smaller beads. Due to this smaller radius of curvature, smaller beads have stronger gradients at their surface than larger beads. The smaller beads also generally have gradients that fall off more rapidly with distance. Further, the magnetic flux at a distance will generally be less for a smaller bead. A mixture of small and larger magnetic beads thus will capture both weakly magnetized materials (i.e., by smaller beads) and strongly magnetized materials that are far from the beads (i.e., by bigger beads).

In most embodiments of the present invention, the magnetic beads are small enough so that they can be manipulated in a microfluidic device.

In one advantageous embodiments, a combination of different types of beads are preferred, e.g., two, three four or five types of beads.

In certain embodiments, the use of one type of magnetic beads, e.g., ferromagnetic beads alone in a microfluidic device may lead to cell death of the recombinant cells due to, e.g. aggregation of cells. In one embodiment of the invention, ferromagnetic beads are used as carrier beads, i.e., their function is to optimize the mixing of cells with capture beads and, in certain embodiments, the recovery of the cells, in particular viable cells. In one embodiment, the carrier beads are non-functionalized. Carrier beads may have a diameter of between 0.1 to 500 μm, preferably between 0.1 and 100 μm, more preferably between 0.2 and 50 μm, between 0.2 and 20 μm, between 0.2 and 10 μm and 0.2 and 5 μm. In a preferred embodiment the diameter is between 1 and 6 μm.

Capture beads do in fact capture the cells of interest. The capture beads are generally functionalized. The capture beads are preferably superparamagnetic beads, which as described above, do not (or insignificantly) clump together and thus allow cells attached to them to stay viable. Carrier beads may have a diameter of between 0.1 to 500 μm, preferably between 0.1 and 100 μm, more preferably between 0.2 and 50 μm, between 0.2 and 20 μm, between 0.2 and 10 μm and 0.2 and 5 μm. In a preferred embodiment the diameter is between 0.5 and 2.5 μm.

The ratio of carrier beads to capture beads may be between 1:1 to 1:50, preferably between 1:5 to 1:40, 1:5 to 1:20, 1:8 to 1:12, 1:9 to 1:11 or about 1:10. As the person skilled in the art will readily understand the absolute amount of ferromagnetic beads and/or non-ferromagnetic beads will depend on the volume of the reaction chamber, the type, composition and size of the magnetic beads and can be empirically determined by the person skilled in the art. The volume of carrier beads per volume reaction chamber may range from 1 μl per 100 μl to 10 μl per 100 μl. For a 50 μl reaction chamber the volume of carrier beads might range, e.g., from 1 μl to 5 μl.

The protein of interest may be a marker protein identifying a stem cells, in particular a cancer stem cell (CSC), including a tissue specific CSC such as leukemia stem cells, or a circulating tumor/cancer or precancerous cell.

In one embodiment, the marker protein may be one or more (e.g. 1, 2, 3, 4, 5, 6, 7, 8, 9, 10, 11, 12, 13, 14, 15, 16, 17, 18, 19, 20, 21, 22 or 23) of stem cell markers from the group consisting of: Lgr5, LGR4, epcam, Cd24a, Cdca7, Axin, CK19, Nestin, Somatostatin, DCAMKL-1, CD44, Sord, Sox9, CD44, Prss23, Spy, Hnf1.alpha., Hnf4a, Sox9, KRT7 and KRT19, Tnfrsf19. The stem cell markers may be tissue specific. For example, pancreatic stem cells or organoids may be characterized by natural expression of one or more (for example 1, 2, 3, 4, 5, 6, 7, 8, 9, 10, 11, 12, 13, 14, or 15 for example, 1, 2, 3 or 4) of: CK19, Nestin, Somatostatin, insulin, glucagon, Ngn3, Pdx1, NeuroD, Nkx2.2, Nkx6.1, Pax6, Mafa, Hnf1b, optionally Tnfrsf19; gastric organoids may be characterized by natural expression of one or more (for example 1, 2, 3 or 4) of: DCAMKL-1, CD44, optionally Tnfrsf19; and crypt-villus organoids may be characterized by expression of one or more or all (for example 1 or 2) of: Sord and/or Prss23. CSC markers include CD19, CD34, CD44, CD90, ALDH1, PL2L, SOX-2 and N-cadherin, whereas they may be depleted or display low amounts of other markers such as CD21, CD24, CD38 or CD133. Leukemia stem cells can be identified as CD34⁺/CD38⁻/CD19+ cells, breast cancer stem cells can be identified as CD44+ but CD24^{low} cells, brain CSCs as CD133+ cells, ovarian CSCs as CD44+ cells, CD117+ and/or CD133+ cells, multiple myeloma CSCs as CD19+ cells, melanoma CSC a CD20+ cells, ependymona CSC as CD133+ cells, prostate CSC as CD44 + cells, as well as cells secreting or displaying at their surface other marker proteins known to be expressed by cancer stem cells. Additional CSC markers include, but are not limited to, CD123, CLL-1, combinations of SLAMs (signaling lymphocyte activation molecule family receptors) and combinations thereof. Additional exemplary markers can be found in U.S. patent application 2008/0118518, which is herein incorporated by reference. Circulating tumor cells, including, but not limited to, cells from solid tumors, may be either from a primary tumor or a metastasis and they can be identified by any marker or combination of markers specific for the tumor.

A “gene of interest” or a “transgene” preferably encodes a protein (structural or regulatory protein). As used herein “protein” refers generally to peptides and polypeptides having more than about ten amino acids, preferably more than 100 amino acids and include complex proteins such as antibodies or fragments thereof. The proteins may be “homologous” to the host (i.e., endogenous to the host cell being utilized), or “heterologous,” (i.e., foreign to the host cell being utilized). While the proteins may be non-substituted, they may also be processed and may contain non-protein moieties such as sugars.

Mammalian cells, which include in the context of the present invention, unmodified or recombinant cells according to the present invention, include, but are not limited to, CSC, CHO (Chinese Hamster Ovary) cells, HEK (Human Embryonic Kidney) 293 cells, stem cells or progenitor cells.

Mammalian recombinant cells, ergo cells that contain a transgene, that express and preferably also display on their surface and in certain embodiments, secrete (shed), high levels of an expression product of a transgene, e.g., a therapeutic protein, or a target protein for a therapeutic molecule, are within the scope of the present invention. In certain embodiments recombinant cells that secrete (shed) a transgene (in addition to expressing and displaying it) are identified/separated from cells that express and display, express and do not display or do not even express the transgene product of interest (see US Patent Publication 20120231449, which is incorporated herein by reference in its entirety). A producer cell refers to a cell that does not only display, but also secretes the transgene product from the cells, i.e., releases the transgene product into its surrounding. Only those cells do indeed “produce” the transgene product, while many other cells may just express or display the transgene product but not secrete efficiently the protein. Thus, they may merely display the transgene protein product at their surface for extended period of time (more than 2 days) without releasing it and are thus not classified as “producer cells” or “high-secreter cells”. Recombinant cells that secrete a transgene product (“producer cells”) at more than 10 but less than 20 picograms of the protein within a day (e.g. picogram/cell/day (pcd)) are considered medium producers, recombinant cells that secrete a transgene product at more than 20, more than 40 or more than 60 pcds are considered high producers and those cells that secrete the transgene product at more than 80 pcds are considered very high producers. Very high producer cells may preferably secrete the transgene product at more than 100 pcds. Cells that hardly produce any expression product (low producer cells) secrete less than 10 pcd. In manual procedures, to identify high, including very high, producer cells that secrete the transgene, secretion, ergo, release, is often interfered with, e.g. via a temperature adjustment (in CHO cells, e.g., keeping the surrounding temperatures below 20 degrees Celsius or 4 degrees Celsius) to allow the secreted protein to be displayed on the surface of the cells from which it is secreted for a sufficient amount of time. Advantageously, due to the rapid capture and release of cells displaying high amounts of transgene product, such temperature adjustments are generally not necessary in the context of the present invention, allowing operation temperatures between 18-40 degrees, or 20-37 degrees Celsius.

The method and device of the present invention preferably can sort more than 100,000, preferably more than 1 million, more preferably 2, 3, 4, 5, 6, 7, 8, 9, or 10 million recombinant cells within less than one hours, preferably less than 20 minutes, even more preferably less than 5 minutes. Producer cells, in particular high and very high producer cells, ergo cells that express and release a transgene product, which are identified and/or separated according to the present invention are preferably more than 90%, more preferably more than 95, 96, 97, 98, 99% or 100% viable after identification and/or sorting. In a preferred embodiment cells displaying the transgene product are selected in a sterile microfluidic device as outlined above.

In the context of the present invention there is only a small subset of mammalian cells expressing at high levels a transgene that is of interest. While a wide array of cells will, after a transfection, express and even display the transgene product, only a small subset are also actual producer cells. As can be seen from the list of the model cells below, only the “F206 cells” are desirable since they actually produce, i.e., release/shed the transgene product within 1 day. Other cells that have equally high expression or even display on their surface, are undesirable since they may not actually be producer cells.

CHO-M (Chinese Hamster Ovary cells) suspension cells: these cells express no IgG and no GFP.

F206 cells: these cells express IgG (IgG+) and GFP (GFP+). These are high IgG displayers and high IgG producers and are very desirable.

BS2 cells: these cells express IgG (IgG+) and BFP (BFP+). These are a medium IgG displayers and medium IgG producers and are non-desirable.

BLC cells: these cells express IgG (IgG+) and BFP (BFP+). These are high IgG displayers and medium IgG producers and are non-desirable.

BHB cells: these cells express IgG (IgG+) and BFP (BFP+). These are very high IgG displayers and medium IgG producers and are non-desirable.

As the person skilled in the art will appreciate, the most valuable cells are producer cells that express and shed/release the transgene product at a rate that is very high. Generally, high producer cells, are cells that in a given sample of cells, e.g., a sample of 5000-10 Mil. cells, preferably 1-5 Mil. cells, are in the upper 40%, preferably the upper 30% or upper 25% (quarter) of the cells of expressing and shedding/releasing a certain product. In absolute terms this means that secrete a transgene product at more than 20, preferably, 40, 60, 80, or even more preferably 100 pcds.

If a cells shall be identified and, preferably selected, that display on their surface, but not necessary secret, it might be of interest to select not only high displayer cells, but also medium and/or low displayer cells. It might be desirable to select cells that are high displayers of one protein, but low displayers of another protein. When labeled with a fluorescent antibody, a high displayer cell may exhibit 100-1000 RLUs (relative light units), while a medium displayer may exhibit 10-100 RLUs, and a low displayer may exhibit 1-10 RLU typically. The RLU are preferably maintained for a period exceeding 48 hrs.

A “microfluidic device”, as used herein, refers to any device that allows for the precise control and manipulation of fluids that are geometrically constrained to structures in which at least one dimension (width, length, height) may be less than 1 mm. Typically, in a microfluidic device, microfluidic channels, and chambers are interconnected. Generally, a microfluidic channel (herein just “channel”) is a true channel, groove, or conduit having at least one dimension in the micrometer (μm), or less than 10⁻³ meter (mm), scale. A “reaction chamber” as used herein, refers to a space within a microfluidic device in which one or more cells may be separated, generally via capture and release via a magnetic bead, from a larger population of cells as the cells are flowed through the device. In one embodiment of the present invention, the reaction chamber is, between 10-500 μl, preferably between 20-200 μl, 30-100 μl or between 40-80 μl or 40-60 μl, including 50 μl in size. A reaction chamber can have many different shapes such a round, square or rhombic.

While the flow of a fluid through a microfluidic channel, can be characterized by the Reynolds number (Re), defined as

Re=LV_{avg}ρ/μ

where L is the most relevant length scale, μ is the fluid viscosity, p is the fluid density, and V_{avg} is the average velocity of the flow, these flow characteristics are disturbed in a reaction chamber and the flow within the reaction chamber can be manipulated by outside sources such as one or more magnetic fields. Due to the small dimensions of channels, the Re is usually much less than 100, often less than 1.0. In this Reynolds number regime, flow is completely laminar and no turbulence occurs. The transition to turbulent flow generally occurs in the range of Reynolds number 2,000.

A reaction chamber has generally an inlet channel and an outlet channel for introducing and removing fluid. A fluid according to the present invention is preferably a liquid medium comprising cells. A microfluidic device and reaction chamber is, for example, disclosed in US patent application publications US 2013/0217144 and US 2010/0159556, which are incorporated herein by reference in their entirety, especially with regard to the configuration of their reaction chambers and set up of magnetic devices (such as four electromagnets) around the reaction chamber, or is commercially available under the trademark MagPhase™ (SPINOMIX). A microfluidic device of the present invention preferably also comprises or is connected to at least one cell sample container which may be loaded with cells to be assessed for, e.g., their protein-producing capabilities and which is connected to the inlet of the reaction chamber; a washing reagent container which is also connected to the inlet of the of the reaction chamber; a waste container which is connected to the outlet of the reaction chamber or combinations thereof.

The microfluidic device of the present invention may also be a cartridge or chip which may be less than 1 cm long and 0.5 cm wide. The microfluidic device might also comprise components that control the movement of the fluids within the device, and may include the magnets, pumps, valves, filters and data processing system components described below. Accordingly, a MagPhase™ (SPINOMIX) device including a cartridge may be considered a microfluidic device.

The movement of fluids in the microfluidic device is based in part on passive forces like capillary forces. However, in the context of the present invention external forces, such as pressure, suction and magnetic forces are additionally applied to transport or mix the fluids of the present invention, e.g., to move a suspension of magnetic beads and recombinant cells within the reaction chamber. The external forces may be driven by a data processing system comprising computational hardware. Readily available computational hardware resources using standard operating systems can be employed and modified according to the teachings provided herein, e.g., something as simple as a personal computer (PC), e.g., Intel x86 or Pentium chip-compatible DOS™, WINDOWS, LINUX, MACINTOSH or SUN) for use in the integrated systems of the invention. Current art in software technology is adequate to allow implementation of the methods taught herein on a computer system. Thus, in specific embodiments, the present invention can comprise a set of logic instructions (either software, or hardware encoded instructions) for performing one or more of the methods as taught herein. For example, software for providing the data and/or statistical analysis can be constructed by one of skill using a standard programming language such as Visual Basic, Fortran, Basic, Java, or the like. Such software can also be constructed utilizing a variety of statistical programming languages, toolkits, or libraries.

The different modes of operation within the microfluidic device, in particular within the reaction chamber, will, as the person skilled in the art will appreciate may be determined by the data processing system. In particular, the data processing system may determine the frequencies and magnetic forces that determine the mode of operation. A succession of operation modes aimed at selecting cells of interest is called an operation circle. One operation circle might last less than 20 mins, less than 15 mins, less than 10 mins or less than 5 mins. The person skilled in the art will appreciate that depending on parameters such a size and shape of the reaction chamber, size, shape and/or material of the magnetic beads or the design of the magnetic devices, the different operation modes described below might need to be adjusted.

MIXING MODE: The mixing mode in the context of the present invention describes an operation mode within the reaction chamber in which particles contained within the fluid are optimally mixed so that capture beads capture cells of displaying a transgene product. The mixing mode might last less than 100, 90, 80, 60, 50 or 40 secs.

More than one type of beads, preferably two types of beads, one of which are carrier beads while the other ones are functionalized capture beads (e.g., ferromagnetic and superparamagenetic beads) may be mixed.

For homogeneous mixing in a reaction chamber, controllable magnetic device (s), e.g., electromagnets arranged around the reaction chamber of a microfluidic device which has been placed in, e.g., a MagPhase® 4 device, are preferably operated in e.g., a circular mode or otherwise alternating mode, at frequencies ranging from 0.1 to 1000 Hertz (Hz) and amperages ranging from 0.1 to 10,000 milliAmperes (mA), but preferably at medium to high frequencies (40 Hz-500 Hz, e.g. 100-150 Hz) and at high magnetic force (200-500 mA, e.g. more preferably 300 mA), so that, e.g., the carrier beads, e.g., ferromagnetic beads, rotate around the chamber near the walls while the capture beads, e.g., superparamagnetic beads, will be dispersed and rotated in a gentle way in the middle of the chamber. To optimize spatial distribution of the superparamagnetic beads, the, e.g., electromagnets are preferably activated consecutively in, e.g., a clockwise rotation and counterclockwise rotation, e.g., for 0.5 s-30 s, e.g., 1 s in clockwise followed by, 0.5 s-30 s, e.g., 1 s in counter clockwise rotation and then, 5-100 s, e.g., 10 s of clockwise rotation. This mixing mode is used for incubating the capture beads with the cells, to capture displaying cells.

CAPTURE MODE: The capture mode in the context of the present invention describes an operation mode within the reaction chamber in the carrier beads capture the capture beads (which have preferably displaying cells attached to them). In one operation circle, the capture mode might last less than 100, 90, 80, 60, 50 or 40 secs.

By continuing the operation in circular fashion but reducing the frequency to, e.g., 0.5 to 40 Hz, e.g., 1 Hz and increasing the magnetic force to e.g., 300 to 600 mA, e.g. 400 mA, the carrier beads will rotate slowly all around the chamber. They will “scan” the chamber volume and capture the capture beads. The remnant magnetization of the carrier beads makes them act as small permanent magnets and the capture beads as well as possibly attached cells will be attracted and bind to them. This prepares for the capture of these complexes into the corners of the chamber described in the next step.

IMMOBILIZATION MODE: The immobilization mode in the context of the present invention describes an operation mode within which complexes of carrier beads, capture beads and cells are localize in the reaction chamber at places that allows further fluid, e.g. in a washing step, to move through the reaction chamber without displacing those complexes from the chamber. In one operation circle, the immobilization mode might last less than 100, 90, 80, 60, 50 or 40 secs.

The magnetic device(s) (poles) of the microfluidic device now operate as permanent magnets, e.g., 2 by 2 at 0 Hz and high magnetic force (e.g., 300 to 600 mA, e.g., 400 mA). The associated carrier and capture beads will be held in the corners of the chamber allowing new solutions (e.g., cells in suspension or washing buffers) to be pumped into the chamber and the solution present in the chamber (undesired cells for example) to be pumped out of the chamber.

BEAD SEPARATION MODE: Following the washing steps, the bead separation is performed as for the mixing mode in step 1, while the high frequency (e.g. 40 Hz-500 Hz, e.g. 100-150 Hz) allows the carrier beads to detach from the capture beads. The beads preferably adopt the same or a similar spatial distribution as in the mixing mode, i.e. the carrier beads circulate near the walls and the capture beads move more slowly around the middle of the chamber.

As the mixing mode, the bead operation mode of one operation circle, might last less than 100, 90, 80, 60, 50 or 40 secs.

RECOVERY MODE: After the beads have been separated, a “bead immobilization” mode is applied. In this mode, the capture beads comprising the cells of interest or just the cells of interest (after loss of their magnetic label), are recovered/eluted from the reaction chamber, while the carrier beads are immobilized within the chamber. In one operation circle, the recovery mode might last less than 80, 60, 50, 40, 30, 20, 10, 5, 4, 3, 2 secs.

The recovery mode may be accomplished with a high frequency of e.g. 40 Hz-500 Hz, e.g. 100 Hz and a medium magnetic force of 30-300 mA, e.g. 100 mA. The high frequency and medium magnetic force is applied for a short time (1-50 s, e.g., 3 s), to ensure that only the carrier beads have enough time to migrate to the chamber's corners due to their strong response to magnetic fields. The, e.g., 100 Hz frequency is applied so that the internal magnetic moments of the capture beads switch direction in response to the magnetic field orientation, which prevents their migration to the chamber's corners. The carrier beads will then stay in suspension in the middle of the chamber allowing their elution and that of the associated cells, by pumping air into the chamber.

The magnetic beads bound to captured cells (e.g., magnetically-labeled cells (MLC)) may be subjected to a further separation. During this separation, the cells separate from the magnetic beads when the magnetic beads lose their attachment to the proteins that mediate attachment to the magnetic bead since the protein is released (secreted) from the cells. Cells losing their attachment to magnetic beads in less than 48 hrs, preferably less than 36 hrs or even more preferably less than 24 hrs, are separated from cells losing their magnetic beads thereafter. The cells losing their magnetic beads in less than 48 hrs, less than 36 hrs or less than 24 hrs are categorized as/tested for high producer/secreter cells or very-high producer/secreter cells.

Experimental Work to Sort Therapeutic Protein-Expressing Cells

The development of a method that allows for the rapid and efficient capture of mammalian cells that secrete high amounts of recombinant therapeutics, as based on the labeling of secreting CHO cells using antibodies conjugated either to a fluorescent molecule or to a biotin molecule or to magnetic microparticles is described herein in detail to illustrate the present invention.

It has been previously shown that placing CHO cells at 20° C. or 4° C. transiently interferes with secretion so that secreted proteins are displayed on the cell surface for up to 24 hours. A fluorescent antibody against the secreted protein can be used to label cells in proportion to their protein display potential (Sen, Hu et al. 1990, Brezinsky, Chiang et al. 2003, Pichler, Hesse et al. 2009).

A similar approach was thus assessed to label CHO cells that do not only display but in fact secrete a therapeutic protein: Cells were labeled with magnetic particles within the reaction chamber of the MagPhase™ selection cartridge. This approach relied on magnetic particles having a diameter 1-10 μm. The controlled magnetic fields and its effect on mixing of the magnetic particles form the basis of the MagPhase™ system, which is designed to mix the cells and particles so that the cells and magnetic particles bind to form magnetically labeled cells, and to sort and immobilize the most highly magnetically labeled cells. Other cells were washed away through MagPhase™ pump-operated channels. Then, highly expressing cells and particles were released from the magnetic field, and finally high producer cells were eluted from the MagPhase™ reaction cartridge into sterile and disposable cell culture dishes. Thanks to the computer-controlled magnetic fields and pumps that operate the microfluidic inlets and outlets of the cartridge, it was possible to adapt this process and optimize it for rapid automated cell handling, so as to allow the processing of populations of more than 100,000, preferably one or more million of cells within minutes, e.g., in less than 30 minutes, in less than 20 minutes, or in less than 10 minutes.

1. Generation of Stably Transfected CHO Cell Lines as References

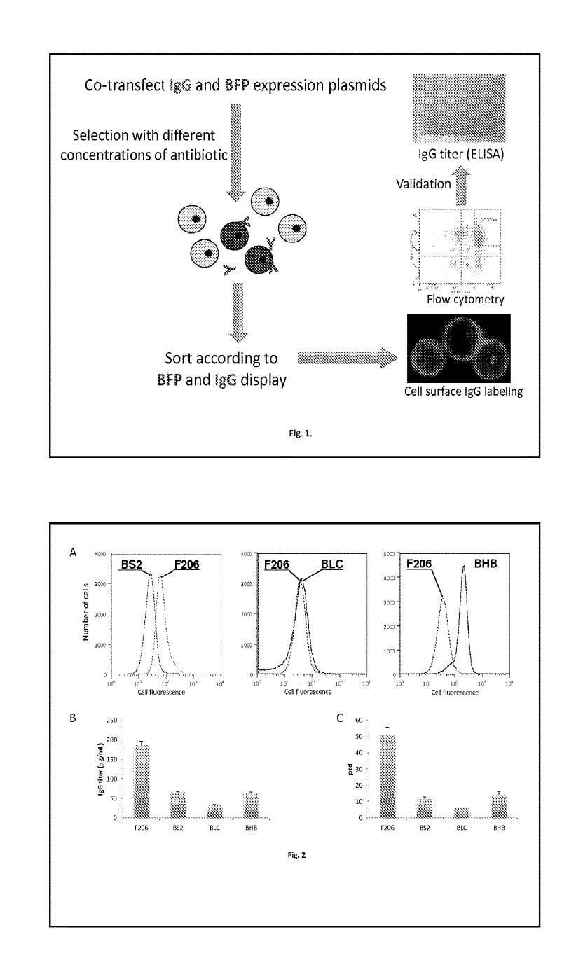

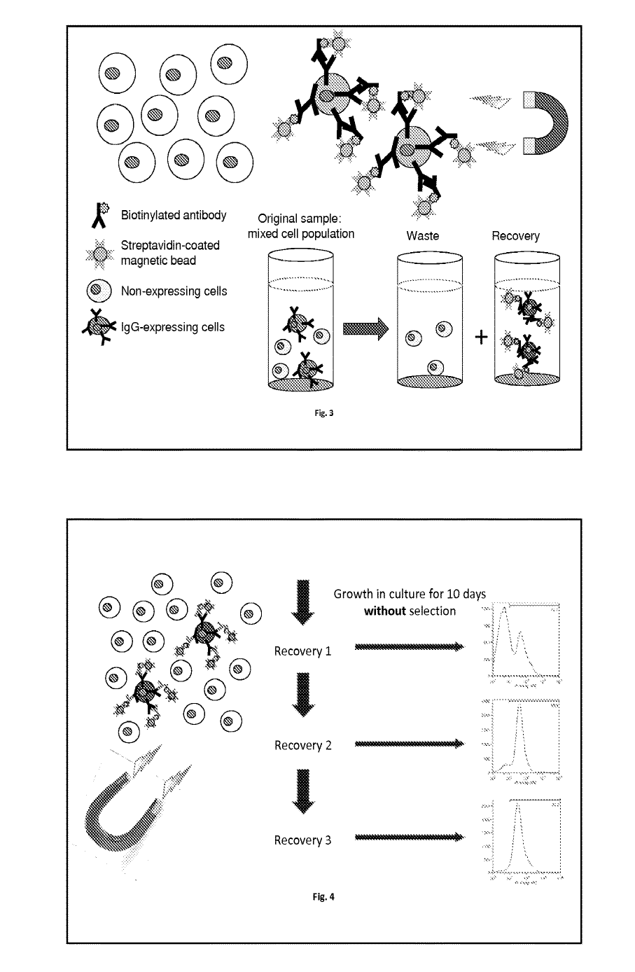

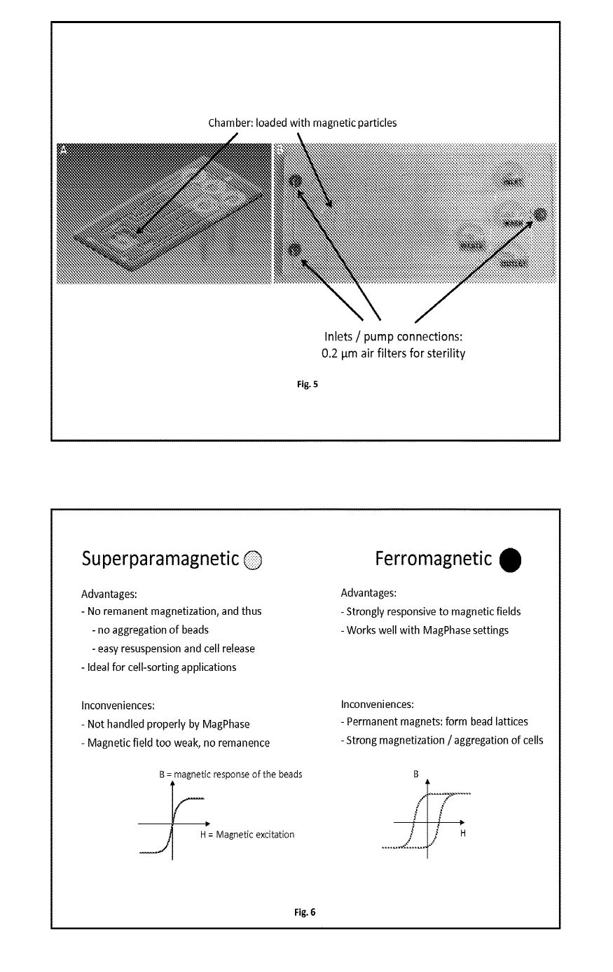

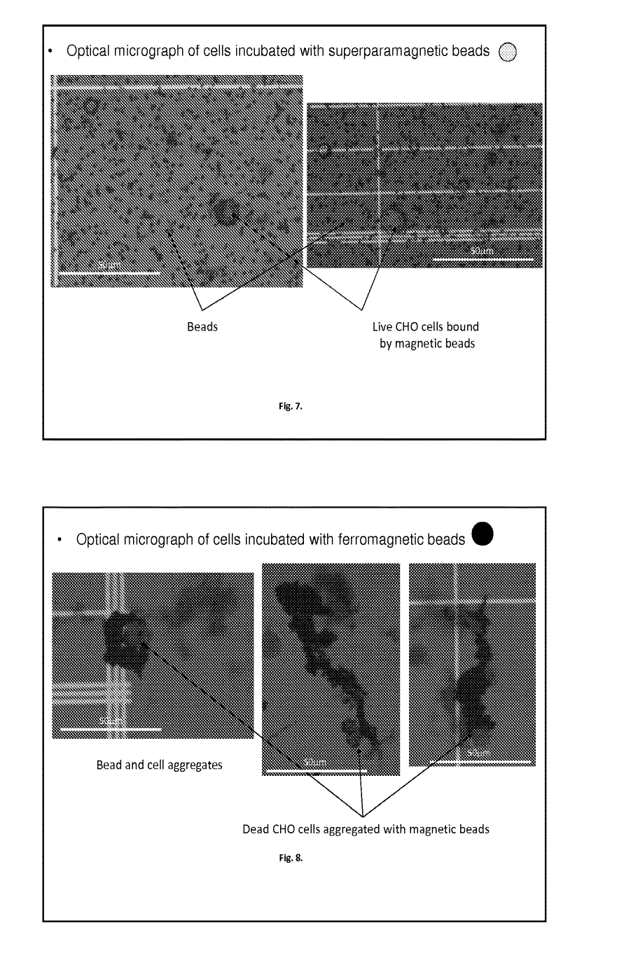

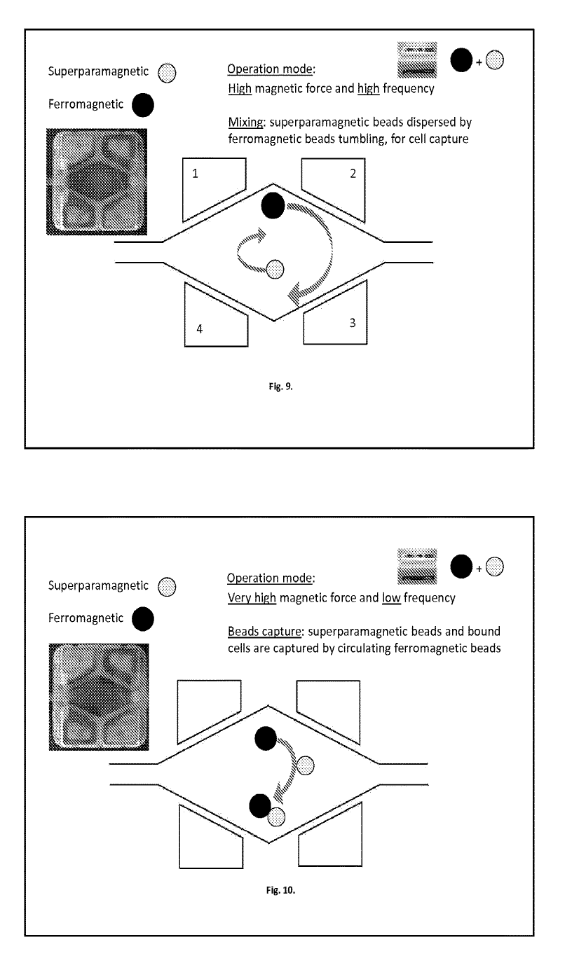

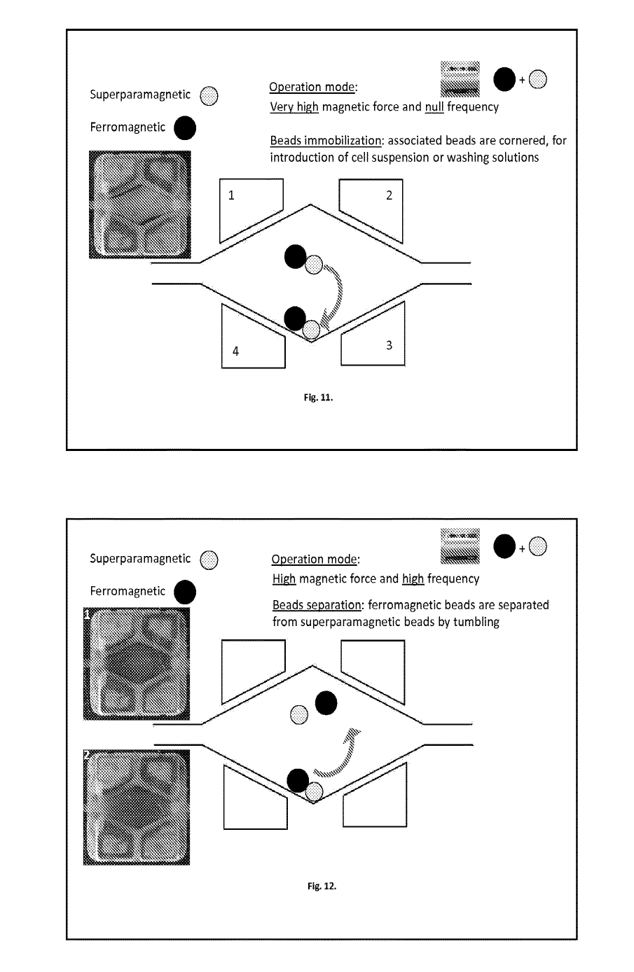

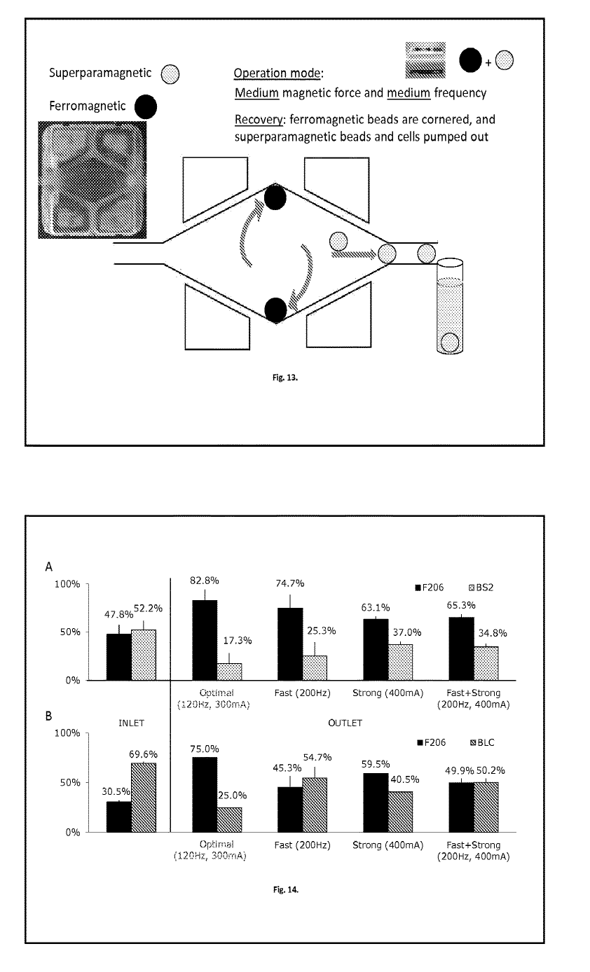

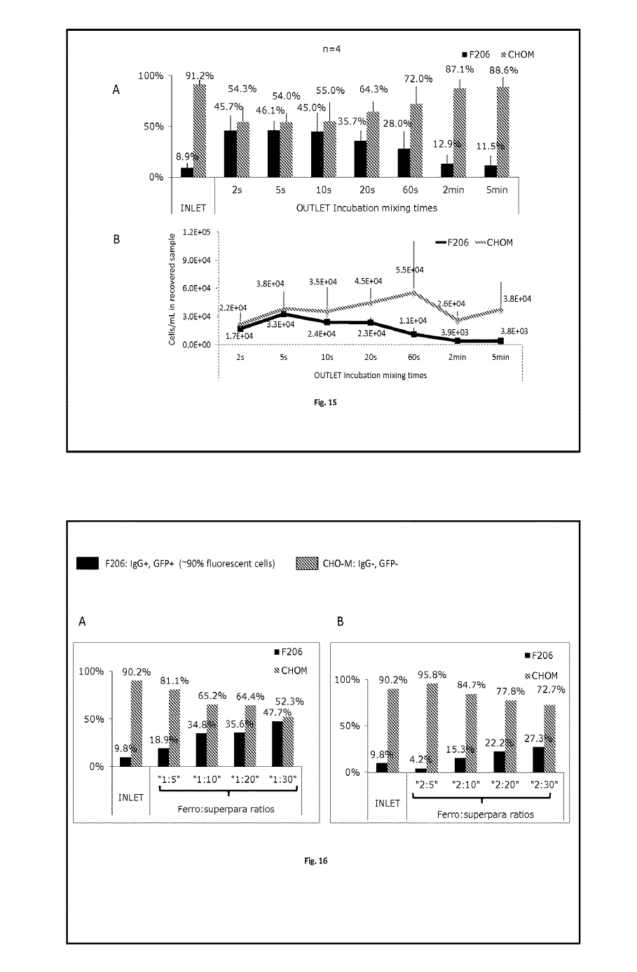

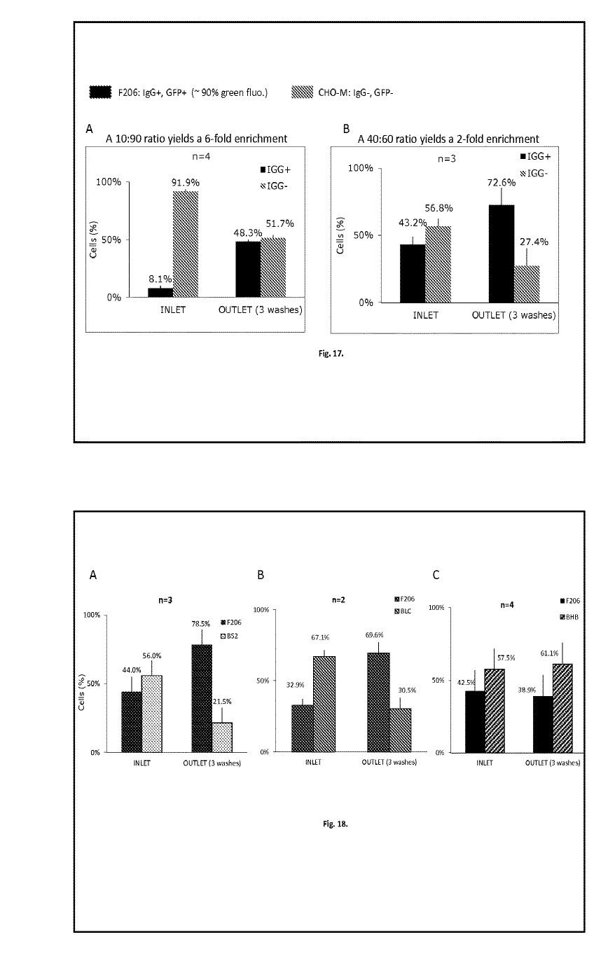

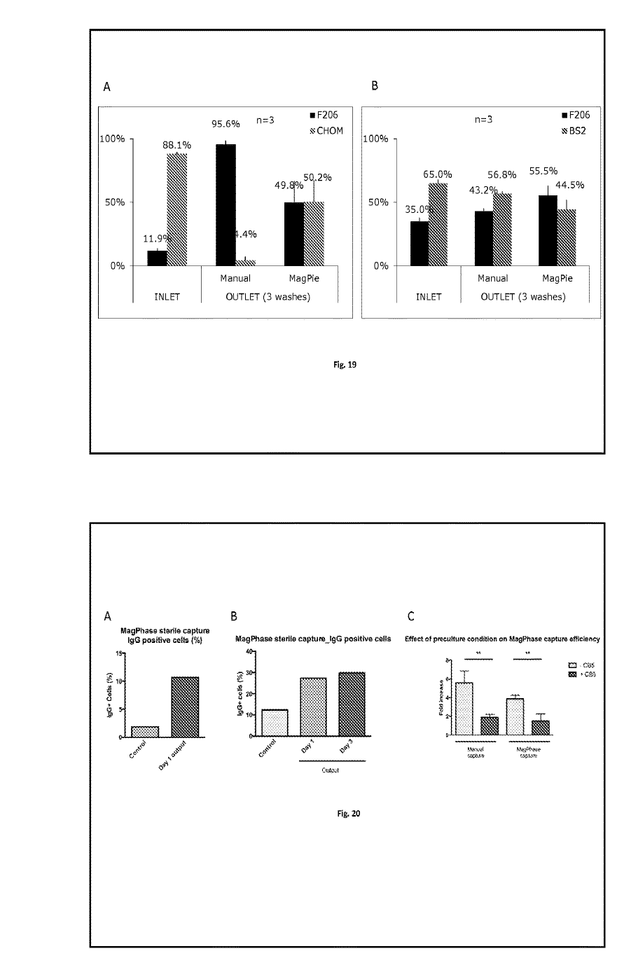

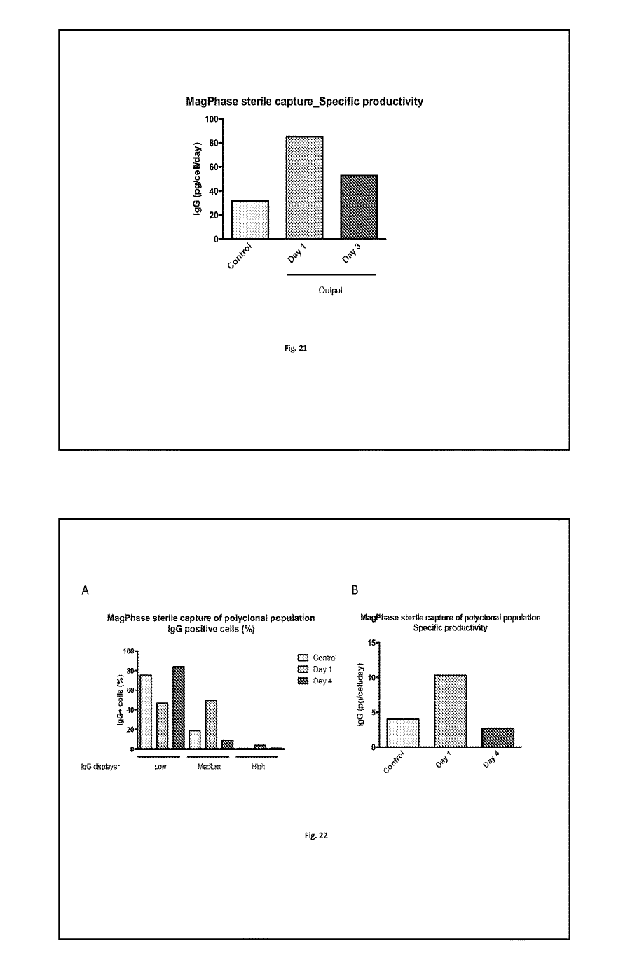

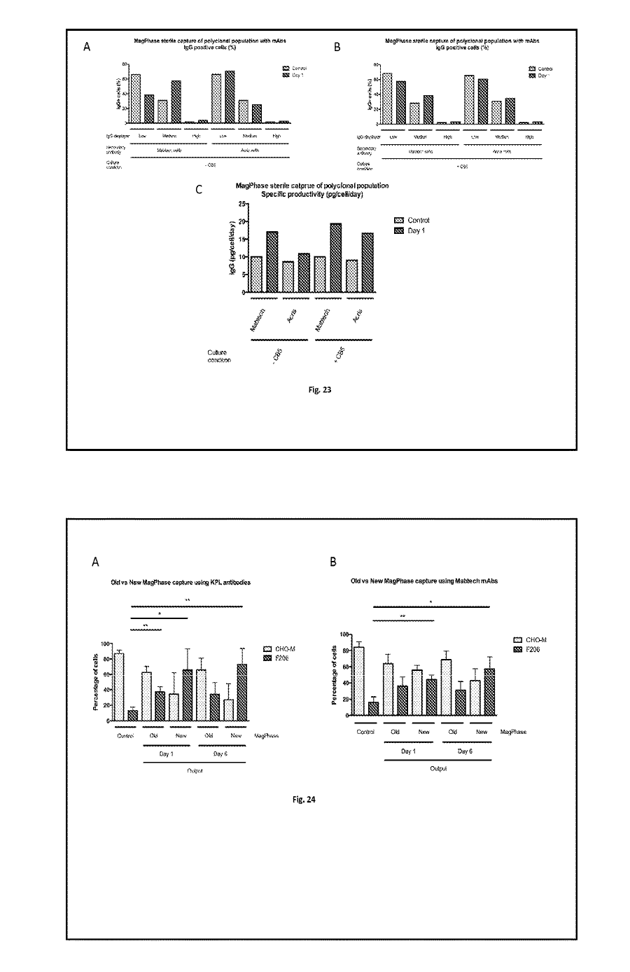

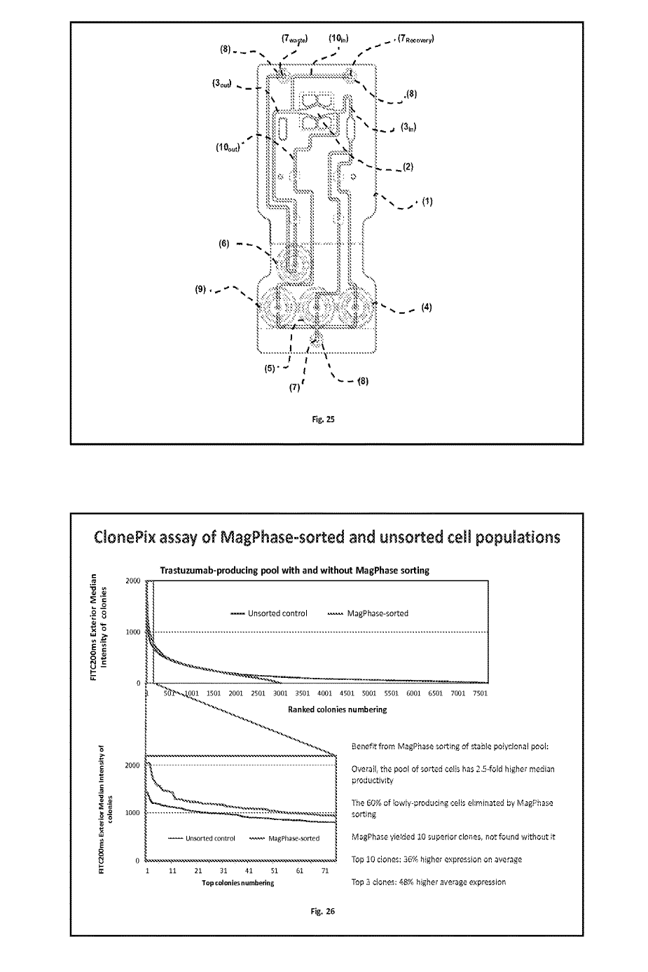

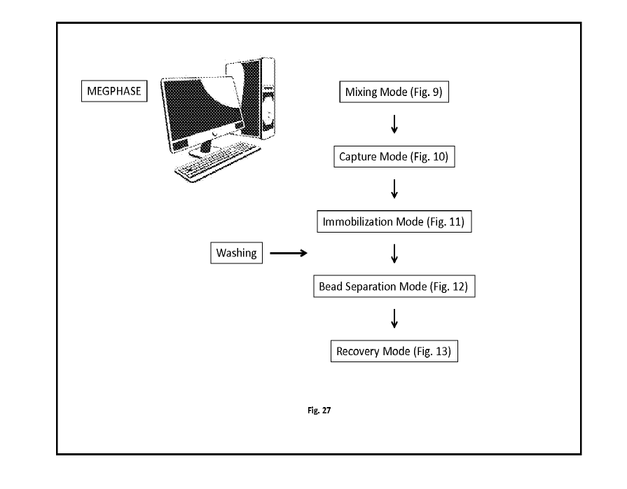

To facilitate the development of the method, and to assess the performance of cell sorting, first reporter cells were designed that would express both a therapeutic protein, namely an immunoglobulin, as well as a fluorescent reporter protein to trace more easily the cells that secrete the antibody. CHO cells were co-transfected with expression vectors for a therapeutic immunoglobulin gamma (IgG) and an antibiotic selection marker, as well as with a plasmid encoding a fluorescent protein, either the ‘enhanced green fluorescent protein’ or the ‘enhanced blue fluorescent protein 2’ (EGFP or eBFP2). Polyclonal populations stably expressing various levels of immunoglobulins were sorted by FACS on the basis of BFP and surface IgG display, and subsequently assessed for IgG production by ELISA (FIG. 1). In parallel, monoclonal CHO cell populations (e.g. cell clones) co-expressing GFP and IgG, or BFP and IgG were selected by limiting dilution. IgG secretion was assessed by ELISA assays. Clones expressing various levels of surface IgG, but with low/medium levels of IgG production were selected as reference cell populations.

The following cell lines were generated and used as references (FIG. 2):

CHO-M suspension cells (no IgG, no GFP)

F206—IgG+, GFP+: a high IgG displayer and HIGH producer, a desired clone.

BS2—IgG+BFP+: a medium IgG displayer, medium IgG producer, a non-desired clone.

BLC—IgG+BFP+: a high IgG displayer, medium IgG producer, a non-desired clone.

BHB—IgG+BFP+: a very high IgG displayer, medium IgG producer, a non-desired clone.

Interestingly, the characterization of these clones indicated that the transient display of a protein, as assessed in FIG. 2A, does not correlate well with the actual secretion rate, as indicated by the titers and specific productivity of the cells (FIGS. 2B and 2C). This indicated that the sorting method should be capable of distinguishing proper protein secretion from the mere display of the protein at the surface of the recombinant cell without release (“shedding”) from the surface.

2. Validation of a Manual Cell Capture Assay with Magnetic Particles

Cell populations expressing either no IgG, or various known levels of IgG, were mixed with defined numbers of cells from the F206 clone secreting high amounts of the Trastuzumab therapeutic IgG and co-expressing GFP. The cells were incubated with a biotin-conjugated secondary antibody conjugated that binds the constant part of human IgGs and subsequently with magnetic microparticles coupled to streptavidin (Dynabeads MyOne T1®, Invitrogen®, #65601) (FIG. 3). A sample of the cells after each wash was retained (referred to as Recovery 1 to 3) and placed in cell culture medium, and cell were grown without selection for 10 days. The cells were then assessed for their surface IgG display, to distinguish non-expressing cells from expressing ones. As shown in FIG. 4, each subsequent wash reduced the percentage of negative cells and after the 3^{rd} wash almost 100% of the positive cells had been recovered.

3. Principles of Antibody-Expressing Cell Capture with the MAGPHASE Microfluidic Device

Once the manual capture process was established, it was implemented in the MagPhase™ device to attempt to capture CHO-M (Selexis®) cells expressing the therapeutic human IgG.

The MagPhase™ equipment had to be adapted for use with single-use cartridges designed to contain microchannels and a 50 μL reaction chamber that was loaded with magnetic beads. FIG. 5 illustrates the employed cartridge design, as specifically optimized for the sterile sorting and recovery of live cells. The cartridge was designed to allow the loading of different solutions (cells in suspension, washing buffers), as well as for the mixing of the magnetic particles, for the washing away of the non-expressing cells, and finally for the elution of the cells that were bound to magnetic beads. The whole process for manual capture was adapted to work in a fully automated manner, to significantly reduce the experimental time and contamination risks.

The manual capture protocol used superparamagnetic beads, which have the advantage of having no remnant magnetization and that behave as non-magnetic particles once the magnetic field has been removed (FIG. 6). Therefore, superparamagnetic beads are in the present context, preferred for cell-sorting applications because the beads can be fully resuspended in solution and the cells can be released from the beads once the antibody is shed from the cell surface, which can occur after about 24 h at 37° C. (FIG. 7).

However, adaptation of the manual sorting protocol to the MagPhase™ device posed a number of problems: The superparamagnetic beads used in the manual capture protocol could not be manipulated by the electromagnetic poles of the MagPhase™ device, because its electromagnets generate lower intensity magnetic fields when compared to hand-held permanent magnets (FIG. 6). Due to their remnant magnetization and strong response to magnetic fields, ferromagnetic beads work well with the MagPhase™ technology and they can be operated in the cartridge chamber in a wide range of operation modes.

Streptavidin-coated ferromagnetic beads, known to function in MagPhase™, were mixed with cells expressing and secreting an IgG that had been labeled with a biotinylated anti-IgG antibody, so as to capture IgG-expressing cells. However, the remnant magnetization of the ferromagnetic microbeads led to their mutual attraction and to the formation of aggregates that trapped cells and killed them (FIG. 8). Furthermore, the cells could not be released from the beads after placing the aggregates in culture (data not shown).

Therefore, a mixture of the two types of magnetic beads was employed. This method allowed the handling of the functionalized superparamagnetic beads in MagPhase™ in the presence of non-functionalized ferromagnetic beads, as shown below.

Initial attempts did not allow the sorting of the best cells, but rather mediated the sorting of cells irrespective of the protein expression levels. Thus, the process had to be improved to retain only highly expressing cells. We evaluated altering various parameters such as the frequencies and magnetic strength of the various MagPhase™ operation modes, the cell and particle titers, the ratio of high producers to the general cell population, the choice of the secondary antibody, the capture conditions, the magnetic mixing speed and duration, and the elution conditions of the magnetically labeled cells.

4. Identification of Magnetic Beads Suitable for MAGPHASE Operation

Various types of commercially available microbeads and bead ratios were tested in the course of these studies, to identify conditions that would give the best results in terms of proper handling by MagPhase™ and in terms of specific and non-specific interactions with CHO cells. These included:

Ferromagnetic Microbeads:
Chemicell™ FluidMAG (with a 5.0 μm diameter)
Chemicell™ SiMAG (with a 1.0 μm or 2.0 μm diameter)

Superparamagnetic Microbeads:
Dynabeads™ M280 2.8 μm
Dynabeads™ MyOne T1 1.0 μm
Ademtech™ 300 nm

Visual distinction of the various types of microbeads within the cartridge was possible, because they display distinct colors, i.e. black for the ferromagnetic beads and light brown for the superparamagnetic Dynabeads™. Visual inspection of the microbeads during MagPhase™ operations suggested that the best volume ratio of ferromagnetic vs. superparamagnetic microbeads under the set conditions is around 1:10 for a homogeneous and gentle mixing of superparamagnetic beads inside the chamber, with a volume of ferromagnetic beads varying from 1 to 5 μL. Using more ferromagnetic beads made it difficult to maintain them close to the walls upon mixing of the superparamagnetic beads. Using less ferromagnetic beads made it difficult to catch the superparamagnetic beads efficiently and to immobilize them on the walls of the cartridge during washes, leading to loss of superparamagnetic bead-associated CHO cells.

The volume of 20-30 μL of packed superparamagnetic beads was based on our protocol for manual cell isolation. The appropriate density of cells was found to be around 1.0×10⁷cells/ml for a chamber volume of 50 μL. The bead to cell ratio used was as recommended by manufacturers, e.g.: the 2.8 μm Dynabeads™ M-280 (Invitrogen, #60210) were used at 6.5×10⁸ beads/mL and the 1.0 μm Dynabeads™ MyOne T1 (Invitrogen, #65601) were at 9×10⁹ beads/mL. Since the MagPhase™ chamber volume is 50 μL and loaded with samples containing 1×10⁷ cells/mL, 20 μL of superparamagnetic beads thus gives a bead: cells ratios of 26:1 for M-280 beads and 360:1 for MyOne T1 beads. Taking into account the diameter and differences in the number of beads, we determined that an equal amount of MyOne T1 beads have nearly twice the surface of M-280 beads, and therefore have a superior capacity than M-280 beads.

Beads were tested using MagPhase™ operation ranges of 0-400 Hz and 0-500 mA. However, optimal conditions were required for the proper handling of the magnetic microbeads by MagPhase™. For instance, under appropriately defined conditions, the ferromagnetic beads circulate around the walls of the chamber and do not localize to the central part of the chamber, while the superparamagnetic beads mix in a gentle way throughout the chamber, with a wide spatial distribution covering the whole chamber volume. When established, these optimized conditions allowed to achieve the “Bead separation mode”, as defined below, in the following section 5. However, optimal conditions were found to vary depending on the microbead type and size, and proper handling by MagPhase™ could only be achieved using specific types of microbeads and operating conditions, as described in the following sections.

Superparamagnetic Beads:

Dynabeads™ M-280 and MyOne T1: Both could be operated in the presence of ferromagnetic beads during the various MagPhase™ operation modes. However, the MyOne T1 beads were chosen because they showed a better spatial repartition. Their weaker magnetization as compared to the M-280 facilitated dissociation from ferromagnetic beads and recovery at the end of the process. Their 1.0 μm size was also found to allow for more specific interactions than the 2.8 μm microbeads for association with CHO cells.

Ademtech™ 300 nm: These beads were not suitable for automated separation, as their magnetization is too weak, making them difficult to be caught and immobilized by the ferromagnetic beads.

Ferromagnetic Beads:

Chemicell™ FluidMAG 5.0 They are magnetically weaker than Chemicell™ SiMAG, yet they provided efficient mixing within a defined range of frequencies and magnetic forces, e.g. 100-200 Hz and 200-300 mA. Optimal mixing conditions could be defined as 150 Hz and 200 mA, as described below, for these ferromagnetic beads. In such conditions, they circulated around the chamber walls and provided a homogeneous and fast spatial repartition of superparamagnetic beads in the mixing or cell capture modes, as illustrated in the following section. However, the Chemicell FluidMAG had to be coated with a layer of starch to reduce their association to non-expressing CHO cells, which bind non-specifically to the silica surface of these beads.

Chemicell™ SiMAG 1.0 μm and 2.0 μm have a stronger magnetism than FluidMAG and thus allow efficient mixing in a wider range of MagPhase™ parameters, e.g. 50-300 Hz and 200-400 mA. Nevertheless, the optimal conditions could be defined as 100 Hz and 300 mA with these microbeads in the “Bead separation mode” and the “Recovery mode”, as illustrated in the following section. In such conditions, these beads circulate near the mixing chamber walls and regroup faster in the chamber's corners than FluidMAG beads, reducing the likelihood of also trapping and immobilizing the superparamagnetic beads along with ferromagnetic beads, and thereby yielding an increased cell recovery when compared with the FluidMAG beads.

5. Setting Up and Optimization of MAGPHASE Operation Parameters

The process for this innovative approach of mixing both ferromagnetic and superparamagnetic particles in the MagPhase™ chamber for the isolation of highly-expressing cells can be described in 5 steps:

Mixing mode (FIG. 9): In this mode, the two types of beads were mixed separately. In order to have homogeneous mixing, one needs to operate the 4 MagPhase™ electromagnets in a circular mode at medium to high frequencies (e.g. 100 Hz) and high magnetic force (e.g. 300 mA). This ensured that the ferromagnetic beads rotate around the chamber near the walls while the superparamagnetic beads will be dispersed and rotated in a gentle way in the middle of the chamber. To achieve an ideal spatial repartition of the superparamagnetic beads, the electromagnets were activated consecutively in a clockwise rotation for 1 s followed by 1 s of anticlockwise rotation and then 10 s of clockwise rotation. The mixing mode is used for incubating capture beads, here the superparamagnetic beads with the cells, to capture expressing cells, and also for the washing steps.

Capture mode (FIG. 10): By keeping the MagPhase™ operation mode in a circular fashion but reducing the frequency to 1 Hz and increasing the magnetic force (e.g. 400 mA), the ferromagnetic beads rotated slowly all around the chamber. They “scanned” the chamber volume and capture the superparamagnetic beads. The remnant magnetization of the ferromagnetic beads makes them act as small permanent magnets and the superparamagnetic beads as well as possibly attached cells will be attracted and bind to them. This prepares for holding these complexes in the corners of the chamber described in the next step.

Immobilization mode (FIG. 11): The electromagnetic poles of the MagPhase™ now operated as permanent magnets 2 by 2 at 0 Hz and high magnetic force (e.g. 400 mA). The associated ferromagnetic and superparamagnetic beads were held in the corners of the chamber allowing new solutions (cells in suspension or washing buffers) to be pumped in and the solution present in the chamber (undesired cells for example) to be pumped out.

Bead separation mode (FIG. 12): Following the washing steps, the bead separation was performed as in the mixing mode in step 1, and the high frequency (100-150 Hz) allowed the superparamagnetic beads to detach from the ferromagnetic ones. The beads adopted the same spatial distribution as in the mixing mode, i.e. the ferromagnetic beads circulate near the walls and the superparamagnetic beads move more slowly around the middle of the chamber.

Recovery mode (FIG. 13): After the beads have been separated, a “bead immobilization” mode is applied with a frequency of 100 Hz and a magnetic force of 100 mA. The high frequency and medium magnetic force is applied for a short time (3 s), to ensure that only the ferromagnetic beads have enough time to migrate to the chamber's corners due to their strong response to magnetic fields. The 100 Hz frequency is applied so that the internal magnetic moments of the superparamagnetic beads switch direction in response to the magnetic field orientation, which prevents their migration to the chamber's corners. The superparamagnetic beads will then stay in suspension in the middle of the chamber allowing their elution and that of the associated cells, by pumping air into the chamber.

An efficient enrichment of IgG-expressing cells requiring specific operation modes that were determined empirically, by optimizing each step and parameter of the MagPhase™ cell capture process. As the person skilled in the art will appreciate these operation modes, once determined, can be readily adjusted, for example when the size of the reaction chamber or the configuration of the electromagnet is changed.

Firstly, the wash mode was optimized. F206 cells mixed with BS2 cells to a 50:50 ratio or with BLC cells to a 30:70 ratio. The cell mixes were incubated with the biotinylated anti-IgG KPL antibody, and the labeled mixes were subjected to MagPhase™ capture with different wash modes, i.e. what was discovered to be the, under the given overall conditions and with the specified equipment, the ‘optimar mode’ (120 Hz, 300 mA), or the ‘Fast’ (200 Hz), ‘Strong’ (400 mA) or ‘Fast+Strong’ (200 Hz, 400 mA) mode. 20 μL of superparamagnetic beads (MyOne T1 Dynabeads™, Streptavidin-coated, 1.0 μm) and 2 μL of ferromagnetic beads (Chemicell™ FluidMAG/MP-D, 5.0 μm, starch coated) were preloaded into the mixing chamber. All other parameters were the default parameters of FIGS. 9 to 13. The Optimal wash mode allowed a 2-fold enrichment of F206 cells from BS2 cells and a 2.5 enrichment of F206 cells from BLC cells (FIG. 14B). Both experiments showed that the ‘Fast’ and/or ‘Strong’ wash mode caused the loss of the desired F206 cells, therefore yielding lower enrichments. This provided the first indications that cells that secrete high levels of the IgG (F206) can be separated from BS2 cells expressing at lower levels, and from the BLC cells that display high levels of the IgG at their surface but do not secrete it efficiently (FIG. 2). This optimal wash mode was used in the following assays.

Secondly, the cell capture time was optimized within the MagPhase™ sorting process. 1 μL of Chemicell™ SiMAG 1.0 μm beads and 20 μL of MyOne T1 Dynabeads™ were preloaded in the mixing chamber. F206 cells were mixed with non-expressing CHO-M cells to a 10:90 ratio. Biotinylated anti-IgG labeled cell mix were subjected to MagPhase™ capture with different time of incubation, ranging from 2 s to 5 min. In terms of percentage of recovered F206 cells from CHO-M cells, 2 s, 5 s and 10 s of incubation time all resulted in 5-fold enrichment (FIG. 15A). Regarding the yields of recovered F206 cells, a 5 s incubation showed the highest yield amongst all tested conditions, which is 2-fold more than the yield obtained with a 2 s incubation, for instance (FIG. 15B). This assay also showed that longer incubation times yielded lower F206 enrichment ratio, most likely due to the increased non-specific binding of CHO-M cells, as seen in FIG. 15B.

Finally, the optimal ratio between ferromagnetic beads and superparamagnetic beads was determined. F206 and CHO-M cells were mixed and pre-labeled as described above. In the mixing chamber, 1 or 2 μL of Chemicell™ SiMAG 1.0 μm, as well as 5 μL, 10 μL, 20 μL or 30 μL of MyOne T1 Dynabeads™ were pre-loaded. As shown in FIG. 16A, the ferromagnetic superparamagnetic beads ratio at 1:30 showed the highest enrichment of F206 cells from CHO-M cells (i.e. 5-fold). When ferromagnetic beads were increased to 2 μL, the F206 cells enrichment was halved when comparing to the results obtained with 1 μL ferromagnetic beads (FIG. 16B). This was likely due to the previously detected non-specific binding of the non-expressing CHO-M cells to ferromagnetic beads.

6. Enrichment of Protein-Expressing Cells Using MAGPHASE

Using the optimized MagPhase™ cell capture procedure, we further analyzed the enrichment potential for high producer cells (i.e. F206 cells) from non-expressing cells (CHO-M cells) as well as from medium, high, or very high IgG displayers (i.e. BS2, BLC and BHB cells, respectively, see FIG. 2).

We first tested MagPhase™ on a F206 and CHO-M cell mix, with F206:CHO-M ratio at 8:92. Using a combination of 20 μL of superparamagnetic beads (MyOne T1 Dynabeads™, Streptavidin-coated, 1.0 μm) and 2 μL of ferromagnetic beads (Chemicell™ FluidMAG/MP-D, 5.0 μm, starch coated) pre-loaded inside the mixing chamber, MagPhase™ could enrich 6-fold F206 cells in its recovery, compared to the input cell mix (FIG. 17A). When the ratio between high-producer F206 cells and non-expressing CHO-M cells was set to 40:60 for the input, the yield of F206 cells was increased to 73% after the MagPhase™ processing, while the fold increase of the F206 cell ratio fell to 2-fold (FIG. 17B). This result can be explained by a saturation of superparamagnetic beads by the F206 cells, suggesting that the upper limit of capture corresponds to about 70% of highly-expressing cells in these conditions.

Using the same ferromagnetic and superparamagnetic beads ratio and MagPhase™ operation modes, we then tested the capacity of MagPhase™ to enrich high-secretor/producer F206 cells from medium and high-displaying BS2, BLC and BHB cells. When F206 cells were mixed with BS2 cells to a ratio of 40:60 in the input, MagPhase™ achieved a 2-fold enrichment of F206 cells (FIG. 18A), similar to the result of F206 enrichment from CHO-M cells, with input ratio at 40:60 (FIG. 17B). Likewise, F206 cells were enriched 2-fold from BLC cells by MagPhase™, when mixed with BLC cells at a 30/70 ratio in the input (FIG. 18B), which correlates well with the higher secretion rate observed from F206 cells. When high secretor/producer F206 cells were mixed with the very high displayer BHB cells at a 40:60 input ratio, MagPhase™ did not enrich for F206 cells (FIG. 18C). This correlated well with the fact that BHB cells display a much higher amount of IgG than F206 cells, even if BHB cells do not secrete higher IgG amounts, and are thus high displayer but not high secretor cells (FIG. 2). Overall, we concluded that MagPhase™ can enrich selectively highly secreting cells among medium or low producer cells, and it also prompted us to further optimize the selectivity of the cell sorting process.

To compare the capture efficiency obtained with the MagPhase™ automated capture relative to the manual capture, biotinylated anti-IgG antibody labeled F206/CHO-M cells (10:90 ratio) and F206:BS2 cells (40/60 ratio) were subjected to MagPhase™ or to the manual capture. In terms of the fold-increase of the F206 cell percentage in the output, MagPhase™ had a 5-fold enrichment of F206 cells from CHO-M cells, compared to a 9-fold enrichment by Manual capture (FIG. 19A). However, in the more useful situation of a mix between higher and medium producer cells, as would be obtained from a stable transfection aiming at isolating high expressor cells, MagPhase™ yielded a significantly better performance than the manual capture for the sorting of F206 from BS2 cells (FIG. 19B). This indicated that MagPhase™ can provide a more selective sorting of higher-producer cells than the manual process, in addition to requiring a much shorter time, and less handling and efforts from the experimenter.

7. MAGPHASE Sterile Capture Enriches IgG-Displaying and High Secretor Cells from Monoclonal Cell Populations

7.1 MAGPHASE Sterile Captures and Captured Cells/Beads Separation Timing Optimization

As it had been established that MagPhase™ is able to enrich antibody high-expressor cells from non-expressing or medium-expressing cells, we first tested whether the capture could be performed in a sterile environment. To this end, the internal liquid handling microfluidic channels of the original MagPhase™ machine were first sterilized under a laminar hood by washing with 16 mL of 8% Javal solution (Reactol™ lab, #99412), 16 mL of 10% Contrad 90 solution (Socochim™, #Decon90) and 32 mL of sterile Milli-Q water. At later stages, and when the optimized process and disposable cartridge design was developed, the cartridges were sterilized by gamma-irradiation (24K Gray) prior to performing the capture.

Inputs of F206 and CHO-M cell mix at 10:90 to 20:80 ratio were used, and subjected these inputs to MagPhase™ sterile capture using the parameters of FIG. 17. The cells and beads recovered from MagPhase™ capture, as well as an aliquot of input cells as control, were placed in culture with 5% of the Cell Boost 5 supplement (CB5, Hyclone, Thermo Scientific™, #SH30865.01) but without antibiotic selection, as our prior tests had demonstrated that the viability of cells eluted from MagPhase™ was increased by the CB5 nutrient mix. MagPhase™-captured cells were separated from the released beads one day after the capture using a hand-held magnet, to recover only the cells that had spontaneously detached from the beads one day after the elution from MagPhase™. Recovered cells were put back in culture without antibiotic selection and with CB5 for 16 days prior to the analysis of the IgG displayed at the surface of the recovered cells (FIG. 20A). This culture time insured the absence of microbial contamination, and thus implied that the capture had been successfully performed in sterile conditions.

7.2 Pre-Culture Condition Optimization for MAGPHASE Sterile Capture

When the input cells were treated with the CB5 feed following the sterile capture using MagPhase™, the cells recovered at Day 1 had a 5.6-fold enrichment of F206 cells when compared to input cells what were not subjected to MagPhase™ sterile capture (FIG. 20A). This enrichment was in line with results obtained with MagPhase™ non-sterile capture of similar input cell mix composition (FIG. 17A). However, when the input F206 and CHO-M cell mix was pre-cultured in presence of 5% of CB5 supplement prior to MagPhase™ sorting, the cells separated at day one only had a 2-fold enrichment of F206 cells (FIG. 20B). When the remaining mix of cells and beads was cultured further until Day 3, prior to the recovery of the cells dissociated from the beads, a similar finding was obtained. This suggested that the feed had interfered with the cell capture when added prior to the cell sorting step.

This possibility was evaluated directly by performing in parallel the manual or MagPhase™ device-mediated capture of F206 cells cultivated with or without the addition of the CB5 feed in the culture medium prior to the sorting process. Again, the presence the CB5 in pre-culture significantly decreases (p<0.01) the fold increase of F206 cells in the output, and this for both capture methods, when compared to a pre-culture performed without CB5 (FIG. 20C). These results indicated that the presence of CB5 in the pre-culture highly likely disturbed the interaction of the F206 cells with the magnetic beads, and therefore the cells should be cultured without CB5 prior to the MagPhase™ capture. The one likely explanation may be that the feed contains biotin, as this should interfere with the interaction of the cell-bound biotinylated antibody with the streptavidin-coated magnetic beads. Another conclusion is that the cells should be cultured in culture media with biotin concentrations that do not exceed 10 μM, and preferably are lower than the 3 μM or 0.1 μM concentrations of biotin that were included in the CDM4CHO or custom cell culture media evaluated in the present application.

It was next assessed whether the MagPhase™-mediated cell capture had enriched the eluted population into cells that secrete high amounts of the therapeutic IgG. This was assessed because the MagPhase™ sorting procedure relies of the transient display of the IgG at the cell surface, but this should not be necessarily associated with a high level of IgG secretion. Indeed, the BHB and BLC cells of FIG. 2 do not secrete very high levels of the IgG when compared to the F206 cells, although they do display the IgG at high or very high levels by cell surface staining. This was assessed by culturing the cells recovered on Day 1 or on Day 3 of FIG. 20B, as well as unsorted control cells, prior to quantifying the secreted IgG in the culture supernatant on Day 10 post-sorting.

The percentage of IgG positive F206 cells were similar at Day 1 or Day 3 post sorting, and they were 2-3 fold higher than the control cells that were not processed by MagPhase™ (FIG. 21A). However, the cells eluted at Day 1 secreted 3-fold more IgG than control cells, while Day 3 cells secreted only about half the amount of the IgG that Day 1 cells secreted (FIG. 21). These results suggested that Day 1 separated cells display high quantity of the IgG and also quickly release it into the media, while the Day 3 separated cells correspond to cells that display well the IgG at their surface, but that do not release it efficiently, and thus are not very good secretor cells. Therefore, elution of the cells at Day 1 after MagPhase™ capture was, in the present setting, the best timing to recover the IgG high secretor cells. Thus, the sorting of high displayer cells using MagPhase™ coupled to the optimal timing of the cell release from the magnetic beads can be used to select cells with the desired property, in this case the secretion in high amounts of the therapeutic protein. Furthermore, it will be apparent to someone skilled in the art that MagPhase™ settings and operation mode may be adapted to recover preferentially medium-, low-, or non-expressing cells.

8. MAGPHASE Sterile Capture Enriches IgG-Secreting Cells from Polyclonal Populations

Above, the MagPhase™ device and method had been tested on mixture of reference monoclonal cells for its efficiency of IgG-expression cell enrichment. We next wanted to determine whether MagPhase™ may also allow the enrichment of high IgG-secreting cells from polyclonal populations containing many widely varying expression levels. To this end, a sterile MagPhase™ capture was performed to capture high IgG-secreting cells from a polyclonal population of cells expressing stably the therapeutic Trastuzumab antibody.

As shown in FIG. 22A, when captured cells were cultured without CB5, there was a 3-fold increase of medium as well as high IgG displayer cells in the cell population eluted at Day 1, when compared to control cells. However, there was no enrichment of medium or high displayer cells from the Day 4 elution. Similar conclusion were obtained in terms of specific productivity as before, in that the best secreting cells were obtained for the Day 1-separated cells, which secreted 2.6-fold more IgG compared to control cells (FIG. 22B), despite the unfavorable presence of the CB5 supplement in the cell pre-culture.

Overall, it was concluded that MagPhase™ can efficiently sort cells in the sterile environment of disposable and single use cartridges, and that it is able to enrich cells that secrete a therapeutic protein at high levels from a heterogeneous polyclonal population. Moreover, adding CB5 in the culture of captured cells, after MagPhase™ cell sorting, further increased the recovery of best secretor cells at Day 1 post capture.

9. MAGPHASE Sterile Captures Using Monoclonal Anti-IgG Antibodies

Since the use of serum-derived polyclonal secondary antibody is not suitable for a pharmaceutical environment, we further explored the feasibility of using biotinylated anti-IgG monoclonal antibodies (mAb) for the MagPhase™ capture process. As shown in FIG. 23A, two distinct monoclonal antibodies could be tested in the MagPhase™ cell capture process. Use of the Mabtech™ monoclonal antibody enriched both medium and high IgG displayer to 2-fold at Day 1 when compared to the control cells, when captured cells were cultured without CB5 (FIG. 23A). When the cells captured using Mabtech™ mAbs were cultured in CB5 containing media, a 1.4-fold and a 1.6-fold increase of medium and high displayer in cells, respectively, was obtained at Day 1 (FIG. 24B). Lower enrichment of medium and high displayer cells were obtained using the Acris mAb during the cell capture. Correspondingly, the IgG specific productivity of the captured cells was higher when using the Mabtech™ mAB, yielding a 2-fold higher productivity than control cells when eluting the cells in presence of the CB5 feed (FIG. 24C). Taken together, these assays indicated that monoclonal antibodies can be used for the MagPhase™-based sterile capture and the enrichment of highly secreting cells from a polyclonal population.

10. New MAGPHASE Enriches Antibody-Expressing Cells

Known versions of the MagPhase™ sorting process involved the sterilization of MagPhase™ by pumping decontamination solutions. In these known processes the microfluidic channels were not single-use either and thus bore contamination risks, rendering them not compatible with cell sorting for pharmacological applications. Presented herein are, among others, a new generation of MagPhase™ machine and cartridges dedicated to the sterile sorting of live cells, allowing all liquid and cell handling procedures to be processed within the contained and defined environment of a single-use sterile cartridge.

After various attempts and improvements in terms of the cartridge constituent material and design, we found that cartridges made in polymethyl methacrylate (PMMA) and with a polycarbonate PC cover film to function well for the sterile cell capture process. The final cartridge design is illustrated in FIG. 5 and FIG. 25.

When KPL polyclonal antibodies were used to label input cell population, the improved MagPhase™ device significantly enriched (5.0-fold increase) F206 cells from CHO-M cells at Day 1, compared to the 2.4-fold enrichment obtained with the original MagPhase™ design (FIG. 24A). Day 6 separated cells had a similar enrichment patent as Day 1 separated cells. Likewise, the improved MagPhase™ also achieved a significant enrichment of F206 cell from CHO-M cells using Mabtech™ mAbs, i.e. 2.8-fold and 3.6-fold enrichment in the Day 1 and Day 6 separated cells, respectively (FIG. 24B). These findings indicated an improved performance of the improved MagPhase™ design, using both the polyclonal KPL antiserum or the Mabtech™ monoclonal antibody.

Overall, a novel microfluidic device including associated cartridges and operating processes are presented that allow the enrichment of cells expressing and secreting higher amounts of a therapeutic protein, and this within sterile, contained, cell viability-compatible and single use vessels, as needed to handle cells that produce therapeutic proteins for human use Given the prior failure to enrich specifically for higher producing cells using previously available approaches, as the manual or semi-automated non-microfluidic previously reported methods could only isolate expressing cells from non-expressing ones, the results were unexpected. Another advantage of the current MagPhase™ setting, when compared to the prior art, is that it is a fully automated and very rapid process (about 5 minutes of automated operations with MagPhase vs at least 45 min of hands-on time for the manual sorting), saving both time and operator's efforts, and reducing dramatically the contamination risks associated with the non-contained cell-sorting environments known in the art.

It will be appreciated that the methods and devices of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### REFERENCES

[01]. Brezinsky, S. C., G. G. Chiang, A. Szilvasi, S. Mohan, R. I. Shapiro, A. MacLean, W. Sisk and G. Thill (2003). “A simple method for enriching populations of transfected CHO cells for cells of higher specific productivity.” J Immunol Methods 277(1-2): 141-155.

[02]. Caron, A. W., C. Nicolas, B. Gaillet, I. Ba, M. Pinard, A. Garnier, B. Massie and R. Gilbert (2009). “Fluorescent labeling in semi-solid medium for selection of mammalian cells secreting high-levels of recombinant proteins.” BMC Biotechnol 9: 42.

[03]. Galbete, J. L., M. Buceta and N. Mermod (2009). “MAR elements regulate the probability of epigenetic switching between active and inactive gene expression.” Mol Biosyst 5(2): 143-150.

[04]. Girod, P. A., D. Q. Nguyen, D. Calabrese, S. Puttini, M. Grandjean, D. Martinet, A. Regamey, D. Saugy, J. S. Beckmann, P. Bucher and N. Mermod (2007). “Genome-wide prediction of matrix attachment regions that increase gene expression in mammalian cells.” Nat Methods 4(9): 747-753.

[05]. Le Fourn V, Girod P A, Buceta M, Regamey A, and Mermod N. (2014). CHO cell engineering to prevent polypeptide aggregation and improve therapeutic protein secretion. Metab. Eng., 21:91-102.

[06]. Ley D, Harraghy N, Le Fourn V, Bire S, Girod P-A, Regamey A, Rouleux-Bonnin F, Bigot Y and Mermod N. (2013). MAR Elements and Transposons for Improved Transgene Integration and Expression. PLoS ONE, 8:e62784.

[07]. Pichler, J., F. Hesse, M. Wieser, R. Kunert, S. S. Galosy, J. E. Mott and N. Borth (2009). “A study on the temperature dependency and time course of the cold capture antibody secretion assay.” J Biotechnol 141(1-2): 80-83.

[08]. on Sen, S., W. S. Hu and F. Srienc (1990). “Flow cytometric study of hybridoma cell culture: correlation between cell surface fluorescence and IgG production rate.” Enzyme Microb Technol 12(8): 571-576.

1. A method for identifying and, optionally selecting, cells displaying a protein of interest on their surface comprising:
a) providing a sample comprising said cells;
b) providing functionalized magnetic beads comprising one or more affinity groups, and optionally carrier beads, wherein said affinity group(s) is/are adapted to bind cells displaying the protein on their surface;
c) mixing the cells with said functionalized magnetic beads and optionally with said carrier beads, wherein said affinity group of the beads binds cells displaying said protein at their surface to produce magnetically-labeled cells (MLCs) having a magnetic label,
d) separating, in an optional at least one washing step, not magnetically-labeled cells from said MLCs, and
e) identifying and, optionally selecting, cells displaying the protein on their surface.

2. The method of claim 1, wherein the protein of interest is a marker protein or a transgene expression product (TEP).

3. The method of claim 1, wherein the cells are recombinant cells and the sample comprises the recombinant cells that were transfected with a transgene, wherein the protein of interest is a transgene expression product (TEP); and wherein the MLCs lose their magnetic label over a time interval after binding to the affinity group(s) and wherein the MLCs are identified, and optionally selected based on the time interval.

4. The method of claim 3, wherein, based on the time interval, recombinant cells secreting the TEP are separated from recombinant cells displaying, but not secreting, the TEP.

5. The method of claim 2, wherein the MLCs that lose the magnetic label in less than 1, 2, 3, 4, 5, 6, 7, 8, 9, 10, 11, 12, 13, 14, 15, 16, 17, 18, 19, 20, 21, 22, 23 hour(s) after binding, in less than 24, in less than 36, in less than 48, in less than 60, in less than 72, in less than 84 or in less than 96 hours after binding are selected.

6. The method of claim 1, wherein the protein of interest is a marker protein identifying a stem cell, in particular a cancer stem cell or a circulating tumor cell.

7. The method of claim 1, wherein the affinity group(s) of the magnetic beads bind(s) the protein directly.

8. The method according to claim 1, further comprising providing at least one linking molecule, wherein the at least one linking molecule binds the affinity group(s) and the protein, linking the magnetic beads to the protein.

9. The method of claim 8, wherein the linking molecule is an antibody or fragment thereof, which is optionally biotinylated.

10. The method according to claim 1, wherein the cells are mixed at a temperature above 20, 24, 26, 28, 30, 32, 34 or 36 degrees.

11. The method according to claim 1, comprising a mixture of said functionalized magnetic beads (capture beads) and carrier beads, wherein the mixture is in a reaction chamber.

12. The method of claim 11, wherein the method further comprises: applying an external magnetic field having an amplitude and a polarity to said reaction chamber, wherein, in said external magnetic field, mixing of the capture beads and the cells displaying the protein is promoted by said carrier beads.

13. The method of claim 12, wherein the capture beads are superparamagnetic beads and the carrier beads are ferromagnetic beads.

14. The method of claim 12, wherein the ratio of said capture beads to said carrier beads is between 2:1 and 50:1, 5:1 and 25:1, between 8:1 and 12:1, or around 10:1.

15. The method of claim 12, further comprising changing the amplitude and/or the polarity to define successive operation modes, wherein said mixing in (c) is performed in a mixing mode and said separating in (d) is performed in a bead separation mode.

16. The method of claim 15, wherein the cells are recombinant cells and wherein the protein expressed on the surface is a TEP and wherein the identifying in (e) is performed by eluting the cells from the reaction chamber that lose their magnetic beads within less than 48 hours, less than 36 or 24 hours after binding.

17. The method of claim 15, wherein in the mixing and bead separation mode, the magnetic field is applied in a circular or alternating mode at 1 Hz-1000 Hz and 0.1 to 10000 mA, or at 40 to 500 Hz and at 200-500 mA.

18. The method of claim 15, wherein the mixing mode and/or bead separation mode each last less than 60 seconds.

19. A cartridge for selecting cells based on their level of display of, and optionally secretion of, a protein from a population of cells comprising the cells displaying, and optionally secreting, said protein, comprising:
a. microfluidic channels,
b. a reaction chamber for mixing magnetic beads in suspension, wherein the reaction chamber has at least one inlet and at least one outlet channel for introducing and removing a fluid into and from said reaction chamber,
c. a cell sample container in fluid communication with the reaction chamber through the inlet channel,
d. at least one washing reagent container in fluid communication with the reaction chamber through the inlet channel,
e. a waste container in fluid communication with the reaction chamber through the outlet channel,
wherein, each container of c-d is further in communication through one of the microfluidic channels to a venting pore comprising an air filtering element.

20. An integrated system for selecting cells based on their level of display of, and optionally secretion of, a protein from a population of cells comprising the cells displaying, and optionally secreting, said protein, comprising:
a. microfluidic channels,
b. a reaction chamber for mixing magnetic beads in suspension; wherein the reaction chamber has at least a first inlet and at least a second outlet channel for introducing and removing a fluid into and from said reaction chamber,
c. a cell sample container in fluid communication with the reaction chamber through the inlet channel,
d. at least one washing reagent container in fluid communication with the reaction chamber through the inlet channel,
e. a waste container in fluid communication with the reaction chamber through the outlet channel, wherein each container of c-d is further in communication through one of the microfluidic channels to an venting pore comprising an air filtering element;
f. one or more devices that create a controllable magnetic field (magnetic field devices=MFDs), in particular one or more electromagnets, arranged around or at the reaction chamber;
g. data processing equipment configured to adjust the magnetic field created by the MFDs within the reaction chamber via frequency and/or amplitude adjustments, wherein each frequency and/or amplitude adjustment defines an operation mode within the reaction chamber.

21. The system of claim 20, wherein the data processing equipment is configured to set a succession of said operation modes comprising a mixing mode, a capture mode, an immobilization mode, a bead separation mode and a recovery mode.

22. The system of claim 21, wherein the data processing equipment is adapted to sets the MFDs to operate:
in a circular or alternating mode at 1-1000 Hz, preferably 40 Hz-500 Hz and at 0.1 to 10,000 mA, preferably 200-500 mA during the mixing and bead separation mode;
in circular or alternating mode at a frequency and amplitude lower than in the mixing mode, such as at 0.5 to 40 Hz and at 300 to 600 mA, during the capture mode;
at 0 Hz and at an amplitude, such as at 300 to 600 mA, during the immobilization mode; and
at an, relative to the immobilization mode, increased frequency, such as between 40 Hz-500 Hz and at a lowered amplitude, such as at 30-300 mA during the recovery mode.

23. The system of claim 20, wherein the reaction chamber comprises a mixture of carrier and capture beads.

24. The cartridge or system according to claim 20, wherein the cartridge further includes a recovery container for receiving magnetically-labeled cells, including magnetically-labelled recombinant cells from the reaction chamber.

25. The cartridge or system according to claim 20, further comprising at least one second inlet and at least one second outlet channel in fluid communication with said reaction chamber, wherein the second inlet channel diverges off the at least one first outlet channel and the second outlet channel diverges off the at least one first inlet channel, wherein the recovery container is in fluid communication with the reaction chamber through the second inlet channel and the second outlet channel is connected to a further venting pore comprising an air filtering element.

26. The system according to claim 25, wherein the air venting pore of the recovery container are connected to a pump for recovering the magnetically-labeled cells within the reaction chamber by pumping air through the venting pore of the recovery container so that the reaction chamber content is flushed into the recovery container through an inlet channel.

27. The cartridge according to claim 19, wherein the reaction chamber volume is between 10 μl and 500 μl.

28. The cartridge according to claim 19, wherein the cartridge is self-contained and disposable.

29. A kit comprising, in one container, a cartridge according to claim 19, wherein capture beads and carrier beads are contained in the reaction chamber or are provided in a further container, and, in a separate container, instructions of how to use the capture beads and carrier beads in the cartridge.

30. The kit of claim 29, wherein the capture beads are superparamagnetic beads and the carrier beads are ferromagnetic beads, wherein the ratio of superparamagnetic beads to ferromagnetic beads is between 2:1 and 50:1.

31. (canceled)

32. An isolated population of cells comprising, optionally recombinant cells secreting a transgene expression product at a level of more than 20, 40, 60, 80 pcd, wherein the isolated population of cells does not contain more than 40% of an original cell population from which the isolated population of cells has been isolated.

33. The isolated population of recombinant cells of claim 32, wherein the transgene secreted is a therapeutic protein.

34. The method of claim 1, wherein the time interval between the mixing of the cells with said functionalized magnetic beads and optionally with said carrier beads, and the identifying and, optionally selecting of cells displaying the protein on their surface is less than 1 hour, less than 30 minutes, less than 20 minutes, less than 15 minutes or less than 10 minutes.
